(19) United States
(12) Patent Application Publication
(10) Pub. No.: US 2024/0043372 A1
(43) Pub. Date: Feb. 8, 2024

(54) SUBSTITUTED AROMATIC COMPOUNDS AND PHARMACEUTICAL COMPOSITIONS THEREOF

(71) Applicant: PHARMACEUTIQUE INGENEW INC., Montreal (CA)

(72) Inventor: Lyne GAGNON, Montreal (CA)

(21) Appl. No.: 18/245,695

(22) PCT Filed: Oct. 5, 2021
(86) PCT No.: PCT/CA2021/051395

§ 371 Date: Mar. 16, 2023

Related U.S. Application Data
(60) Provisional application No. 63/088,266, filed on Oct. 6, 2020.

(51) Int. Cl. C07C 235/34; C07C 39/11; C07C 215/68; C07C 33/20; A61K 45/06; A61P 17/02; A61P 35/00

(52) U.S. Cl. CPC C07C 235/34; C07C 39/11; C07C 215/68; C07C 33/20; A61K 45/06; A61P 17/02; A61P 35/00

(57) ABSTRACT

Compounds of Formula I or II, methods of preparation and of use thereof. Formula I has a core aromatic group with substituents as follows: Formula (I) wherein G₁ is —(CH₂)ₙC(R₁)(R₂)OH, —(CH₂)ₙ; —CHO, —(CH₂)ₙC(O)NR₁R₂, —(CH₂)ₙCH (R₁)NR₁R₂, —(CH₂)ₙC(O)OR₃, —(CH₂)ₙ—CH(R₁)O—R₃, or —(CH₂)ₙC(O)R₃; G₂ and G₄ are independently H, OH, F, or Cl, where G₂ can also be NH₂; G₃ and G₅ are independently (H, F, Cl, OH, —(CH₂)ₙ-optionally substituted heterocycle, —(CH₂)ₙ-optionally substituted phenyl, —(CH₂)ₙC₃H₅, optionally substituted C₁-C₆ alkyl, optionally substituted C₂-C₆ alkenyl, —C(O)—R₃, or CH(OH)—R₃; and G₆ is H, F, Cl, OH, —(CH₂)ₙ-optionally substituted heterocycle, —(CH₂)ₙ-optionally substituted phenyl, or (CH₂)ₙCOOH, wherein ⋅n is an integer selected from 0 to 5, ⋅R₁ and R₂ are independently selected from H and optionally substituted C₁-C₆ alkyl group, and ⋅R₃ is an optionally substituted C₁-C₆ alkyl group or when present on G₁ forms a lactone with the core aromatic group, or a pharmaceutically acceptable salt thereof.

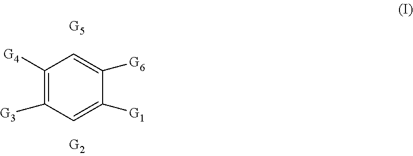

### CROSS REFERENCE TO RELATED APPLICATIONS

The present application claims the benefit of U.S. provisional patent application Ser. No. 63/088,266 filed on Oct. 6, 2020, by Lyne Gagnon. The contents of the above-referenced document are incorporated herein by reference in their entirety.

### TECHNICAL FIELD

The present disclosure relates to compounds and their pharmaceutical uses. More particularly, the disclosure relates to substituted aromatic compounds, to processes for their manufacturing, to composition including same and to their use for the prevention and/or treatment of various diseases and conditions in a subject.

### BACKGROUND

Cancer: Cancer refers to more than one hundred clinically distinct forms of the disease. Almost every tissue of the body can give rise to cancer and some can even yield several types of cancer. Cancer is characterized by an abnormal growth of cells which can invade the tissue of origin or spread to other sites. In fact, the seriousness of a particular cancer, or the degree of malignancy, is based upon the propensity of cancer cells for invasion and the ability to spread. That is, various human cancers (e.g., carcinomas) differ appreciably as to their ability to spread from a primary site or tumor and metastasize throughout the body. Indeed, it is the process of tumor metastasis which is detrimental to the survival of the cancer patient. A surgeon can remove a primary tumor, but a cancer that has metastasized often reaches too many places to permit a surgical cure. To successfully metastasize, cancer cells must detach from their original location, invade a blood or lymphatic vessel, travel in the circulation to a new site, and establish a tumor.

There are many types of cancer treatment. The types of treatment that you have will depend on the type of cancer you have and how advanced it is. Some people with cancer will have only one treatment. But most people have a combination of treatments, such as surgery with chemotherapy and/or radiation therapy. You may also have immunotherapy, targeted therapy, stem cell/bone marrow treatment, hormone therapy, laser or hyperthermia therapy. The twelve major cancers are prostate, breast, lung, colorectal, bladder, non-Hodgkin's lymphoma, uterine, melanoma, kidney, leukemia, ovarian, and pancreatic cancers. Some cancers can have a high percentage of 5-year survival. However, other cancers can have a low percentage (below 25%) of 5-year survival, this is the case of glioblastoma, heart, esophageal, liver and bile duct, pancreas, lung, gallbladder, mesothelioma, diffuse intrinsic pontine glioma, and acute myelomonocytic leukemia.

Often, cancers may be more or less effectively treated with chemotherapeutic agents (also referred to as cytotoxic drugs). However, chemotherapeutic agents suffer from two major limitations. First, chemotherapeutic agents are not specific for cancer cells and particularly at high doses, they are toxic to normal rapidly dividing cells. Second, with time and repeated use cancer cells develop resistance to chemotherapeutic agents thereby providing no further benefit to the patient. Subsequently, other treatment modalities have been investigated to address the limitations imposed by the use of chemotherapeutic agents. Alternative, well-studied treatment options are surgery, radiation and immunotherapy. However, these treatments also have serious limitations especially in more advanced cancers. Thus, for example, surgery is limited by the ability to completely remove extensive metastases, radiation is limited by the ability to selectively deliver radiation and penetrate cancer cells and immunotherapy (e.g., use of approved cytokines) is limited by the balance between efficacy and toxicity. For this reason, other relatively newer therapeutic approaches are under study. These approaches include the use of protein kinase inhibitors (not selective and therefore toxic and still prone to drug resistance), anti-angiogenesis agents (limited efficacy and toxicity) and gene therapy (no significant success to date), hyperthermia therapy (limited to certain cancers). Therefore, a need still exists for novel compounds which are efficacious (e.g., reduce tumor size and/or spread of metastases) and have reduced toxicity for the treatment of cancer.

As an example, the first treatment step for glioblastoma is surgery to remove as much tumor as possible. Glioblastoma has the capacity to extensively invade and infiltrate normal surrounding brain tissue that makes complete resection impossible. After surgery, radiation therapy is used to treat any residual visible tumor on imaging and any microscopic tumor cells in the surrounding region in an attempt to prevent recurrence. Chemotherapy is often given at the same time as radiation, and often given alone after the combination of chemotherapy and radiotherapy is completed. In children, chemotherapy may be used to delay the need for radiotherapy. However, it is very difficult to treat glioblastoma due to several factors: the tumor cells are very resistant, and the brain is susceptible to conventional therapies. Further many drugs can not cross the blood-brain barrier (BBB) to act on the tumor and the brain has a very limited capacity to repair itself. There is a need for compounds who cross the BBB or a delivery system for such compounds in the brain which is able to slowly release the anticancer compounds.

Fibrosis-related diseases: Fibrosis refers to the formation or development of excess fibrous connective tissue in an organ or tissue that can occur as a part of the wound-healing process in damaged tissue. It may be viewed as an exaggerated form of wound healing that does not resolve itself.

Fibrosis can occur on the skin but it can also occur in internal organs such as the kidney, heart, lung, liver and brain. In the case of organs, fibrosis will often precede sclerosis and subsequent shutdown of the affected organ. Of course, the most common consequence of complete organ failure is death. Thus, for example, pulmonary fibrosis is a major cause of morbidity and mortality. It is associated with the use of high dose chemotherapy (e.g., bleomycin) and bone marrow transplantation. Idiopathic pulmonary fibrosis (IPF) is a lung fibrotic disease for which the median survival is four to five years after the onset of symptoms. Currently there are two compounds, pirfenidone and nintedanib, approved for human needs. However, these compounds reduce slightly the disease progression and have serious side effects. Therefore, the need exists for compounds that are useful for the treatment of fibrotic diseases.

Renal fibrosis is the common pathway underlying the progression of chronic renal injury to end-stage renal disease. The kidney is a structurally complex organ that performs a number of important functions: excretion of the waste products of metabolism, regulation of body water and salt, maintenance of acid balance, and excretion of a variety of hormones and autocoids. Diseases of the kidney are complex but their study is facilitated by dividing them by their effects on four basic morphologic components: glomeruli, tubules, interstitium, and blood vessels. Unfortunately, some disorders affect more than one structure and the anatomic interdependence of structures in the kidney implies that damage to one almost always secondarily affects the others. Thus, whatever the origin, there is a tendency for all forms of renal disease ultimately to destroy all four components of the kidney, culminating in chronic renal failure. For instance, in autoimmune diseases such as diabetes mellitus, the kidneys are prime targets to suffer tissue damage or lesions. Nephrectomy, or kidney removal, a procedure which is sometimes performed on patients with kidney cancer (e.g., renal cell carcinoma), may negatively impact kidney function in the remaining kidney. Chemotherapy and immunosuppressive therapy are also a source of harmful effects to the kidneys. Therefore, there exists a need for drugs with a good safety profile which can be administered to patients with kidney disease. There is also a need for pharmaceutical compounds which can prolong kidney health or protect it from deterioration to the point at which the kidney can no longer function.

Myeloproliferative disorders are associated with bone marrow fibrosis and erythropoiesis failure resulting in extramedullary haematopoiesis (Agarwal et al. Bone marrow fibrosis in primary myelofibrosis: pathogenic mechanisms and the role of TGF-β. Stem Cell Investig. 2016; 3:5). Myelofibrosis (MF) is a fatal disorder of the bone marrow which disturbs the normal production of the blood cells in the body. This results in massive scarring in the bone marrow leading to severe anemia, fatigue, weakness and usually an enlarged liver and spleen. Currently, there is only one approved drug, Incyte/Novartis' Jakafi (ruxolitinib), for the treatment of MF, and other conventional therapies used in MF are off-label. However, none of these drugs are curative, and the only potentially curative intervention is allogeneic stem cell transplant, which is available to a very small percentage of eligible patients because of the high risk of morbidity and mortality. Therefore, there is a huge unmet need for the treatment of MF.

Liver fibrosis such as non-alcoholic fatty liver disease/non-alcoholic steatohepatitis (NAFL/NASH) is also in need of a treatment to reduce, prevent or reverse liver fibrosis.

Inflammation: Immune Mediated inflammatory Disease (IMID) refers to any of a group of conditions or diseases that lack a definitive etiology but which are characterized by common inflammatory pathways leading to inflammation, and which may result from, or be triggered by, a dysregulation of the normal immune response. Autoimmune disease refers to any of a group of diseases or disorders in which tissue injury is associated with a humoral and/or cell-mediated immune response to body constituents or, in a broader sense, an immune response to self. Current treatments for autoimmune disease can be broadly classified into two groups: those drugs which dampen or suppress the immune response to self and those drugs which address the symptoms that arise from chronic inflammation. In greater detail, conventional treatments for autoimmune diseases (e.g., primarily arthritis) are (1) Nonsteroidal Anti-Inflammatory Drugs (NSAIDs) such as aspirin, ibuprofen, naproxen, etodolac, and ketoprofen; (2) Corticosteroids such as prednisone and dexamethasone; (3) Disease-Modifying Anti-Rheumatic Drugs (DMARDs) such as methotrexate, azathioprine, cyclophosphamide, cyclosporin A, Sandimmune™, Neoral™, and FK506 (tacrolimus); (4) Biologicals such as the recombinant proteins Remicade™, Enbrel™ and Humira™. While numerous therapies are available, conventional treatments are not routinely efficacious. More problematic is the accompanying toxicity which often prohibits the long-term use necessary with a chronic disease. Therefore, there is a need for compounds that are useful for the treatment of inflammatory-related diseases, including chronic and non-chronic autoimmune disease.

Oxidative stress: Oxidative stress is caused by an imbalance between the production of reactive oxygen species and a biological system's ability to readily detoxify the reactive intermediates or easily repair the resulting damage. Although reactive oxygen species can be beneficial, as they are used in cell signaling and by the immune system they are also involved in many diseases. Therefore, a need still exists for compounds which can help maintain a proper balance in levels of reactive oxygen species in order to prevent damage to the cell or its components that may be caused by toxic effects of such reactive species.

Metabolic disorders: Metabolic diseases such as diabetes, obesity, non-alcoholic steatohepatitis (NASH) and non-alcoholic fatty liver disease (NAFLD) pose prominent threats to health worldwide and are expected to continue to become more prominent. In 2015, nearly 10% of the American population had diabetes. In addition, more than one-third of American adults have obesity.

While there have been various attempts in the art to use substituted aromatic compounds for treatment and/or prevention of fibrosis or fibrosis-related diseases (e.g., WO 2014/138906), or phenylketone carboxylate compounds for diabetes or diabetes-related disorders (e.g., WO 2012/097428), or medium-chain length fatty alcohols compounds as stimulators of hematopoiesis (e.g., WO 2006/086871), the commercialization results obtained so far have remained unsatisfactory. There still remains the need for compounds, pharmaceutical compositions and treatment methods that alleviate at least some of the pre-commercialization problems observed with the at least some of the compounds described in the afore-mentioned art.

### SUMMARY

This Summary is provided to introduce a selection of concepts in a simplified form that are further described below in the Detailed Description. This Summary is not intended to identify key aspects or essential aspects of the claimed subject matter.

In one broad aspect, the present disclosure relates to a compound according to Formula I having a core aromatic group with substituents as follows:

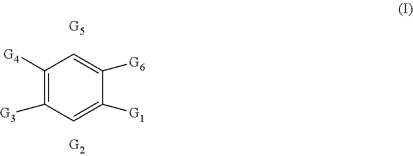

wherein
G₁ is —(CH₂)ₙC(R₁)(R₂)OH, —(CH₂)ₙ—CHO, —(CH₂)ₙC(O)NR₁R₂, —(CH₂)ₙCH (R₁)NR₁R₂, —(CH₂)ₙC(O)OR₃, —(CH₂)ₙ—CH(R₁)O—R₃, or —(CH₂)ₙC(O)R₃;
G₂ is H, NH₂, OH, F, or Cl, preferably H, NH₂, or OH;
G₃ is H, F, Cl, OH, —(CH₂)ₙ-optionally substituted heterocycle, —(CH₂)ₙ-optionally substituted phenyl, —(CH₂)ₙC₃H₅, optionally substituted C₁-C₆ alkyl, optionally substituted C₂-C₆ alkenyl, —C(O)—R₃, and CH(OH)—R₃; preferably optionally substituted C₅ alkyl, optionally substituted C₅ alkenyl, C(O)—(CH₂)ₙ—CH₃ or CH(OH)—(CH₂)ₙ—CH₃ wherein n is 3; more preferably optionally substituted C₆ alkyl, optionally substituted C₆ alkenyl, C(O)—(CH₂)ₙ—CH₃ or CH(OH)—(CH₂)ₙ—CH₃ wherein n is 4; even more preferably optionally substituted C₅ alkyl, optionally substituted C₆ alkyl, optionally substituted C₅ alkenyl, optionally substituted C₆ alkenyl; yet even more preferably optionally substituted C₅ alkyl or optionally substituted C₅ alkenyl; particularly preferably optionally substituted C₅ alkyl or optionally substituted C₆ alkyl; more particularly preferably optionally substituted C₅ alkyl;
G₄ is H, OH, F or Cl, preferably H or OH, more preferably OH;
G₅ is H, OH, F, Cl, —(CH₂)ₙ-optionally substituted heterocycle, —(CH₂)ₙ-optionally substituted phenyl, —(CH₂)ₙC₃H₅, optionally substituted C₁-C₆ alkyl, optionally substituted C₂-C₆ alkenyl, —C(O)—R₃, or CH(OH)—R₃; preferably optionally substituted C₅ alkyl, optionally substituted C₅ alkenyl, C(O)—(CH₂)ₙ—CH₃ or CH(OH)—(CH₂)ₙ—CH₃ wherein n is 3; more preferably optionally substituted C₆ alkyl, optionally substituted C₆ alkenyl, C(O)—(CH₂)ₙ—CH₃ or CH(OH)—(CH₂)ₙ—CH₃ wherein n is 4; even more preferably optionally substituted C₅ alkyl, optionally substituted C₆ alkyl, optionally substituted C₅ alkenyl, optionally substituted C₆ alkenyl; yet even more preferably optionally substituted C₅ alkyl or optionally substituted C₅ alkenyl; particularly preferably optionally substituted C₅ alkyl or optionally substituted C₆ alkyl; more particularly preferably optionally substituted C₅ alkyl; and
G₆ is H, F, Cl, OH, —(CH₂)ₙ-optionally substituted heterocycle, —(CH₂)ₙ-optionally substituted phenyl, or (CH₂)ₙCOOH,
wherein
   n is an integer selected from 0 to 5, preferably 1 to 5, more preferably 1 to 3;
   R₁ and R₂ are independently selected from H and optionally substituted C₁-C₆ alkyl group, and
   R₃ is an optionally substituted C₁-C₆ alkyl group or when present on G₁ forms a lactone with the core aromatic group,
or a pharmaceutically acceptable salt thereof.

In one broad aspect, the present disclosure relates to a use of the compound herein described or a pharmaceutical salt thereof for treatment or prevention of cancer, inflammatory-related disease, oxidative stress, pain, metabolic disorder or a fibrotic-related disease in a subject.

In one broad aspect, the present disclosure relates to a use of the compound herein described or a pharmaceutical salt thereof for use in manufacturing a medicament for treatment or prevention of cancer, inflammatory-related disease, oxidative stress, pain, metabolic disorder or a fibrotic-related disease in a subject.

In one broad aspect, the present disclosure relates to a method for treatment or prevention of cancer, inflammatory-related disease, oxidative stress, pain, metabolic disorder or a fibrotic-related disease in a subject, comprising administering to the subject the compound herein described or a pharmaceutical salt thereof.

In specific embodiments, the herein described use and methods may further include one or more of the following features:
G₃ can be C₅ alkyl, C₅ alkenyl, —C(O)—(CH₂)₃—CH₃ or —CH(OH)—(CH₂)₃—CH₃;
G₃ can be C₆ alkyl, C₆ alkenyl, —C(O)—(CH₂)₄—CH₃ or —CH(OH)—(CH₂)₄—CH₃;
can be C₅ alkyl, C₆ alkyl, C₅ alkenyl, or C₆ alkenyl;
G₃ can be C₅ alkyl or C₅ alkenyl;
G₃ can be C₅ alkyl or C₆ alkyl;
G₃ can be C₅ alkyl;
G₃ can be —(CH₂)ₙ-optionally substituted phenyl;
the phenyl can be substituted with an optionally substituted C₁-C₆ alkyl;
G₃ can be CH₃(CH₂)ₓ—C₆H₄—(CH₂)_{y}— wherein x+y=4 or 5, and wherein y can be an integer selected from 0 to 5;
G₃ can be —(CH₂)ₙ-optionally substituted heterocycle;
the heterocycle has from 1 to 3 heteroatoms selected from nitrogen, oxygen, and sulfur;
the heterocycle can be a non-aromatic monocyclic or polycyclic ring;
the heterocycle can be an aromatic ring;
G₅ can be H, OH, F, —CH₂Phe, —CH₂—C₃H₅, C₄-C₆ alkyl, —(CH₂)ₙCH═CH, or —CH═CH(CH₂), wherein n can be 2 or 3;
G₁ can be
   —(CH₂)ₙCH(CH₃)OH;
   —(CH₂)ₙ—CH—O—CH₃;
   —(CH₂)ₙCH(O)NH₂;
   —(CH₂)ₙC(O)R₃;
   —C(CH₃)₂OH;
   —CH(F)—OH;
   —CF₂—OH;
   —C(O)CH₃;
   —(CH₂)ₙCOOH;
   —CH(CH₃)COOH;
   —C(CH₃)₂COOH;
   —CH(F)—COOH;
   —CH₂C(O)OR₃;
   —(CH₂)ₙC(O)R₃; or
   —CF₂—COOH,
   or pharmaceutically acceptable salt thereof;
G₁ can be —(CH₂)ₙC(R₁)(R₂)OH;
G₁ can be —(CH₂)ₙ—CHO;
G₁ can be —(CH₂)ₙC(O)NR₁R₂;
G₁ can be —(CH₂)ₙCH (R₁)NR₁R₂;
G₁ can be —(CH₂)ₙC(O)OR₃;
G₁ can be —(CH₂)ₙ—CH(R₁)O—R₃;
G₁ can be —(CH₂)ₙC(O)R₃;
the compound can be:
   2-(2-hydroxypropyl)-4,6-dipentylphenol;
   4-benzyl-2-(2-hydroxypropyl)-6-pentylphenol;
   2,4-dibenzyl-6-(2-hydroxypropyl)phenol;
   2-benzyl-6-(2-hydroxypropyl)-4-pentylphenol;
   2,4-bis(3-cyclopropylpropyl)-6-(2-hydroxypropyl)phenol;
   2-(2-hydroxy-3,5-dipentylphenyl)acetamide;
   2-(5-benzyl-2-hydroxy-3-pentylphenyl)acetamide;
   2-(3-benzyl-2-hydroxy-5-pentylphenyl)acetic acid;
   2-(3,5-bis(3-cyclopropylpropyl)-2-hydroxyphenyl)acetic acid;
   2-(2-hydroxy-3,5-dipentylphenyl)acetamide;
   2-(5-benzyl-2-hydroxy-3-pentylphenyl)acetamide;
   2-(3,5-dibenzyl-2-hydroxyphenyl)acetamide;
   2-(3-benzyl-2-hydroxy-5-pentylphenyl)acetamide;
   2-(3,5-bis(3-cyclopropylpropyl)-2-hydroxyphenyl)acetamide;
   2-(2-hydroxy-3,5-dipentylphenyl)acetaldehyde;
   2-(5-benzyl-2-hydroxy-3-pentylphenyl)acetaldehyde;
   2-(3,5-dibenzyl-2-hydroxyphenyl)acetaldehyde;
   2-(3-benzyl-2-hydroxy-5-pentylphenyl)acetaldehyde;
   2-(3,5-bis(3-cyclopropylpropyl)-2-hydroxyphenyl)acetaldehyde;
   1-(2-hydroxy-3,5-dipentylphenyl)propan-2-one;
   1-(5-benzyl-2-hydroxy-3-pentylphenyl)propan-2-one;
   1-(3,5-dibenzyl-2-hydroxyphenyl)propan-2-one;
   1-(3-benzyl-2-hydroxy-5-pentylphenyl)propan-2-one;
   1-(3,5-bis(3-cyclopropylpropyl)-2-hydroxyphenyl)propan-2-one;
   methyl 2-(2-hydroxy-3,5-dipentylphenyl)acetate;
   methyl 2-(5-benzyl-2-hydroxy-3-pentylphenyl)acetate;
   methyl 2-(3,5-dibenzyl-2-hydroxyphenyl)acetate;
   methyl 2-(3-benzyl-2-hydroxy-5-pentylphenyl)acetate;
   methyl 2-(3,5-bis(3-cyclopropylpropyl)-2-hydroxyphenyl)acetate;
   2-(2-methoxypropyl)-4,6-dipentylphenol;
   4-benzyl-2-(2-methoxypropyl)-6-pentylphenol;
   2,4-dibenzyl-6-(2-methoxypropyl)phenol;
   2-benzyl-6-(2-methoxypropyl)-4-pentylphenol; or
   2,4-bis(3-cyclopropylpropyl)-6-(2-methoxypropyl)phenol,
   or pharmaceutically acceptable salt thereof;
the compound can be:

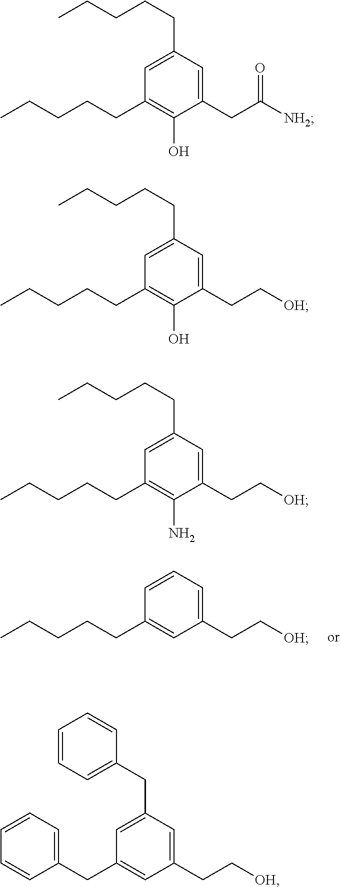

or pharmaceutically acceptable salt thereof.
the pharmaceutically acceptable salt can be a salt such as sodium, potassium, lithium, ammonium, calcium, magnesium, manganese, zinc, iron, olamine, meglumine, lysine, tromethamine, or copper salt, preferably sodium, potassium, magnesium, calcium or lithium salt, more preferably sodium salt;
the pharmaceutically acceptable salt can be a salt such as acetate, benzoate, besylate, bromide, carbonate, citrate, edisylate, estolate, fumarate, gluconate, hippurate, iodide, maleate, mesylate, methylsulfate, napsylate, oxalate, pamoate, phosphate, stearate, succinate, sulfate, tartrate, tosylate, or chloride salt.
the pharmaceutically acceptable salt can be an inorganic or organic salt.
the treatment of cancer includes inhibition of tumor growth, cell proliferation, tumor cell migration, or metastasis in the subject;
the compound can be for use in combination with an anticancer therapy in the subject;
the anticancer therapy can be chemotherapy or ionizing radiations;
the ionizing radiations are selected from X-rays, ion beams, electron beams, gamma-rays, and radiations from a radioactive isotope;
the compound can be for use in combination with an anticancer agent;
the anticancer agent can be temozolomide, abraxane, decarbazine, doxorubicin, daunorubicin, cyclophosphamide, busulfex, busulfan, bleomycin, alectinib, melphalan, pamidronate, bevacizumab, carbozantinib, vinblastine, docetaxel, prednisolone, ifosphamide, dexamethasone, vincristine, bleomycin, etoposide, topotecan, mitomycine, irinotecan, taxotere, taxol, 5-fluorouracil, folfirinox, methotrexate, gemcitabine, cisplatin, carboplatin, chlorambucil, beribucin, or tyrosine kinase inhibitors;
the cancer can be bladder cancer, breast cancer, colorectal cancer, kidney cancer, melanoma, non-Hodgkin's lymphoma, lung, liver, leukemia, glioblastoma, ovarian cancer, pancreatic cancer, prostate cancer or uterine cancer;
the cancer can be glioblastoma or melanoma, and wherein the compound can be for administration in combination with chitosan for in situ treatment of recurrence of cancer;
the fibrosis-related disease can be a lung, kidney, liver, heart, or skin fibrosis-related disease;
the compound can be used for reducing proliferation or progression of fibrotic tissue in fibrotic-related disease;
the subject can be human;
the compound or pharmaceutically acceptable salt thereof can be formulated in a form suitable for enteral, mucosal, parenteral or topical administration;
the compound or pharmaceutically acceptable salt thereof can be formulated in a controlled release composition.

In one broad aspect, the present disclosure relates to a method for manufacturing an alcohol form of the aromatic compound as described herein, the method comprising (a) incubating a mixture of a starting aromatic compound and an olefinic boronic ester derivative having a number of carbons corresponding to the desired G₃ substituent, wherein the starting aromatic compound has an ester at G₁ and a halogen at G₅, under suitable conditions to obtain a first intermediate compound having a structure comprising the ester at G₁ and an alkene chain at G₃ having the number of carbons corresponding to the desired G₃ substituent, (b) incubating the first intermediate compound under suitable conditions to obtain a second intermediate compound having an alkyl chain at G₃ having the number of carbons corresponding to the desired G₃ substituent, and (c) incubating the second intermediate compound under suitable conditions to obtain the alcohol form of the aromatic compound.

In specific embodiments, the herein described method for manufacturing may further include one or more of the following features:
The suitable conditions under step (a) comprises incubating in presence of a first palladium-containing catalyst.
The first palladium-containing catalyst includes Pd(PPh₃)₄.
The suitable conditions under step (a) further comprises incubating in presence of Na₂CO₃
The suitable conditions under step (a) further comprises incubating for a period of from about 16 h to about 18 h.
The suitable conditions under step (a) further comprises incubating at a temperature of about 90° C.
The suitable conditions under step (b) comprises incubating in presence of a second palladium-containing catalyst.
The second palladium-containing catalyst includes Pd(OH)₂
The suitable conditions under step (b) comprises incubating under 5 bar H₂ pressure.
The suitable conditions under step (c) comprises incubating in presence of a reducing agent.
The reducing agent comprises lithium aluminium hydride.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF FIGURES

The patent or application file contains at least one drawing executed in color. Copies of this patent or patent application publication with color drawing(s) will be provided by the Office upon request and payment of the necessary fee. A detailed description of specific exemplary embodiments is provided herein below with reference to the accompanying figures in which:

FIG. 1 shows a non-limiting histogram illustrating data showing anticancer activity of a representative compound relatively to that one of carboplatin (“Carbo”) on human glioblastoma U87 in CAM Avatar model, in accordance with embodiments of the present disclosure.

FIG. 2 shows a non-limiting histogram illustrating data showing anticancer activity of representative compounds relatively to that one of Sorafenib on human renal carcinoma Caki cells in Avatar CAM model, in accordance with embodiments of the present disclosure.

FIG. 3 shows a non-limiting histogram illustrating data showing synergistic anticancer activity of carboplatin (“Carbo”) and a representative compound on a carboplatin-resistant PDX glioblastoma (GBM20-75), in accordance with embodiments of the present disclosure.

FIG. 4 shows a non-limiting histogram illustrating data showing inhibition of growth of PDX-IPF lung fragment by a representative compound relatively to that one of setogepram, in accordance with embodiments of the present disclosure.

FIG. 5 shows a non-limiting histogram illustrating data as well as photographs (in colour) showing inhibition of collagen deposition in PDX-IPF lung fragment by a representative compound relatively to that one of setogepram, in accordance with embodiments of the present disclosure

FIG. 6 shows non-limiting histograms illustrating data showing inhibition of IL-6 release from LPS-stimulated PBMC by representative compounds relatively to that one of setogepram, in accordance with embodiments of the present disclosure.

FIG. 7 shows non-limiting histograms illustrating data showing inhibition of MCP-1 release from LPS-stimulated PBMC by representative compounds relatively to that one of setogepram, in accordance with embodiments of the present disclosure.

FIG. 8 shows non-limiting histograms illustrating data showing inhibition of TNFα release from LPS-stimulated PBMC by representative compounds relatively to that one of setogepram, in accordance with embodiments of the present disclosure.

FIG. 9 shows non-limiting histograms illustrating data showing inhibition of IL-1β release from LPS-stimulated PBMC by representative compounds relatively to that one of setogepram, in accordance with embodiments of the present disclosure.

In the figures, exemplary embodiments or results are illustrated by way of example. It is to be expressly understood that the description and figures are only for the purpose of illustrating certain embodiments and are an aid for understanding. They are not intended to be a definition of the limits of the invention.

### DETAILED DESCRIPTION

A detailed description of one or more embodiments of the present disclosure is provided below along with accompanying figures that illustrate principles of the present disclosure. The invention is described in connection with such embodiments, but the invention is not limited to any embodiment. The scope of the present disclosure is limited only by the claims. Numerous specific details are set forth in the following description in order to provide a thorough understanding of the present disclosure. These details are provided for the purpose of non-limiting examples and the invention may be practiced according to the claims without some or all these specific details. For the purpose of clarity, technical material that is known in the technical fields related to the invention has not been described in detail so that the invention is not unnecessarily obscured.

The present inventor has through R&D work surprisingly and unexpectedly discovered that alcohol, aldehyde, amine, amide, ester, ether, lactone and/or ketone forms of substituted aromatic compounds (i.e., herein described Formula I) as well as acceptable salt thereof are particularly suited for commercial pharmaceutical applications.

For example, such pharmaceutical applications include manufacturing of pharmaceutical compositions, therapeutic uses and methods thereof, for example for preventing and/or treating cancer, inflammatory-related disease, oxidative stress, pain, metabolic disorder or fibrotic-related diseases. For example, it has been discovered that the herein described compounds demonstrated at least one or more of the following advantageous characteristics: improved pharmacokinetics, improved half-life, improved toxicity and/or reduction of undesirable metabolites relative to known structures.

Without being bound by any theory, it is believed that the herein described G₁ group and/or the herein described G₂-G₆ substituents on the aromatic core, affords one or more of the herein described advantageous characteristics to the compounds of the present disclosure. Such advantageous characteristics were unexpected and surprising in view of the known art. For instance, it is believed that the herein described G₁ group afford to the compounds of the present disclosure superior pharmacokinetic/safety profile compared to similar compounds but having a carboxylic acid at G₁, for example leading to less formation of glucuronide metabolites, which represents an advantageous commercial realization at least because such metabolites, and especially the acyl-glucoronide, are known to induce idiosyncratic adverse events and are therefore not well regarded by health agencies and regulators. For instance, it is believed that the herein described G₁ group afford to the compounds of the present disclosure superior biological activity compared to similar compounds but having a carboxylic acid at G₁. This was surprising, unexpected and counter-intuitive at least because, the carboxylic acid functional group plays a cardinal role in drug design and this functional group is often part of the pharmacophore of diverse classes of therapeutic agents (Hajduk et al., J. Med. Chem. 2000; 43:3443-3447). Indeed, a large number (>450) of carboxylic acid-containing drugs have been marketed worldwide, including widely used nonsteroidal anti-inflammatory drugs (NSAIDs), antibiotics, anticoagulants, and cholesterol-lowering statins, among others. The acidity, combined with the ability to establish relatively strong electrostatic interactions and hydrogen bonds, is often brought up as being the reasons this functional group is believed to be a key determinant in drug-target interactions.

### A) Compounds

In a broad aspect, the present disclosure relates to a compound of Formula I having a core aromatic group with substituents as follows:

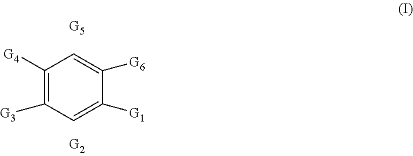

wherein
G₁ is —(CH₂)ₙC(R₁)(R₂)OH, —(CH₂)ₙ—CHO, —(CH₂)ₙC(O)NR₁R₂, —(CH₂)ₙCH (R₁)NR₁R₂, —(CH₂)ₙC(O)OR₃, —(CH₂)ₙ—CH(R₁)O—R₃, or —(CH₂)ₙC(O)R₃;
G₂ is H, OH, NH₂, F, or Cl, preferably H, NH₂, or OH;
G₃ is H, F, Cl, OH, —(CH₂)ₙ-optionally substituted heterocycle, —(CH₂)ₙ-optionally substituted phenyl, —(CH₂)ₙC₃H₅, optionally substituted C₁-C₆ alkyl, optionally substituted C₂-C₆alkenyl, —C(O)—
R₃, and CH(OH)—R₃; preferably optionally substituted C₅ alkyl, optionally substituted C₅ alkenyl, C(O)—(CH₂)ₙ—CH₃ or CH(OH)—(CH₂)ₙ—CH₃ wherein n is 3; more preferably optionally substituted C₆ alkyl, optionally substituted C₆ alkenyl, C(O)—(CH₂)ₙ—CH₃ or CH(OH)—(CH₂)ₙ—CH₃ wherein n is 4; even more preferably optionally substituted C₅ alkyl, optionally substituted C₆ alkyl, optionally substituted C₅ alkenyl, optionally substituted C₆ alkenyl; yet even more preferably optionally substituted C₅ alkyl or optionally substituted C₅ alkenyl; particularly preferably optionally substituted C₅ alkyl or optionally substituted C₆ alkyl; more particularly preferably optionally substituted C₅ alkyl;
G₄ is H, OH, F or Cl, preferably H or OH, more preferably OH;
G₅ is H, OH, F, Cl, —(CH₂)ₙ-optionally substituted heterocycle, —(CH₂)ₙ-optionally substituted phenyl, —(CH₂)ₙC₃H₅, optionally substituted C₁-C₆ alkyl, optionally substituted C₂-C₆alkenyl, —C(O)—R₃, or CH(OH)—R₃; preferably optionally substituted C₅ alkyl, optionally substituted C₅ alkenyl, C(O)—(CH₂)ₙ—CH₃ or CH(OH)—(CH₂)ₙ—CH₃ wherein n is 3; more preferably optionally substituted C₆ alkyl, optionally substituted C₆ alkenyl, C(O)—(CH₂)ₙ—CH₃ or CH(OH)—(CH₂)ₙ—CH₃ wherein n is 4; even more preferably optionally substituted C₅ alkyl, optionally substituted C₆ alkyl, optionally substituted C₅ alkenyl, optionally substituted C₆ alkenyl; yet even more preferably optionally substituted C₅ alkyl or optionally substituted C₅ alkenyl; particularly preferably optionally substituted C₅ alkyl or optionally substituted C₆ alkyl; more particularly preferably optionally substituted C₅ alkyl; and
G₆ is H, F, Cl, OH, —(CH₂)ₙ-optionally substituted heterocycle, —(CH₂)ₙ-optionally substituted phenyl, or (CH₂)ₙCOOH,
   wherein
n is an integer selected from 0 to 5, preferably 1 to 5, more preferably 1 to 3;
R₁ and R₂ are independently selected from H and optionally substituted C₁-C₆ alkyl group, and
R₃ is an optionally substituted C₁-C₆ alkyl group or when present on G₁ forms a lactone with the core aromatic group,
or a pharmaceutically acceptable salt thereof.

In particular, the functional group at position G₁ does not include a carboxylic acid.

The term “optionally substituted heterocycle” refers to a cyclic compound that has atoms of at least two different elements as members of its ring(s). Preferably, the heterocycle is a five- or six-membered ring. Preferably, the heterocycle has from 1 to 3 heteroatoms selected from nitrogen, oxygen, and sulfur. The heterocycle may be an “heterocycloalkyl”, i.e., a non-aromatic monocyclic or polycyclic ring comprising carbon and hydrogen atoms and at least one heteroatom, or may be an “heteroaromatic”, i.e., an aromatic ring containing at least one heteroatom as part of the aromatic ring. Examples of heterocycloalkyl groups include but without being limited to aziridinyl, pyrrolidinyl, pyrrolidino, piperidinyl, piperidino, piperazinyl, piperazino, morpholinyl, morpholino, thiomorpholinyl, thiomorpholino, tetrahydrofuranyl, tetrahydrothiofuranyl, tetrahydropyranyl, and pyranyl. Examples of heteroaromatic groups include but without being limited to pyridine, furan, tiophene, cytosine, and indole. The heterocycle can be unsubstituted or substituted with one or two suitable substituents, for example with an optionally substituted C₁-C₆ alkyl.

Non-limitative examples of a —(CH₂)ₙ-optionally substituted heterocycle may include a group where the heterocycle is substituted with an optionally substituted C₁-C₆ alkyl. In a non-limiting example, the —(CH₂)ₙ-optionally substituted heterocycle may include a group such as CH₃(CH₂)ₓ-heterocycle-(CH₂)_{y}— where x+y=3, 4 or 5, and where y is an integer selected from 0 to 5, preferably 1 to 5, more preferably 1 to 4. Non-limiting examples where y is an integer of from 0 to 5 may include any one of the following (where (CH₂)_{y} is on the far right of the illustrated structures and is shown connected to the rest of Formula I with the wavy bond):

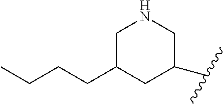 [SMILES: *C1CNCC(CCCC)C1]

(i.e., where y is 0 and x+y is 3),

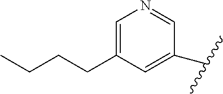 [SMILES: *c1cncc(CCCC)c1]

(i.e., where y is 0 and x+y is 3), and the like.

The —(CH₂)ₙ-optionally substituted phenyl may include a group where the phenyl is substituted with an optionally substituted C₁-C₆ alkyl. In a non-limiting example, the —(CH₂)ₙ-optionally substituted phenyl may include a —(CH₂)ₙ-substituted phenyl group such as CH₃(CH₂)ₓ—C₆H₄—(CH₂)_{y}— where x+y=3, 4 or 5, and where y is an integer selected from 0 to 5, preferably 1 to 5, more preferably 1 to 4. Non-limiting examples where y is an integer of from 0 to 5 may include any one of the following (where (CH₂)_{y} is on the far right of the illustrated structures and is shown connected to the rest of Formula I with the wavy bond):

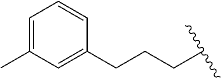 [SMILES: *CCCc1cccc(C)c1]

(i.e., where y is 3 and x+y is 3);

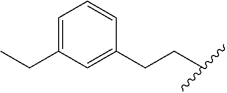 [SMILES: *CCc1cccc(CC)c1]

(i.e., where y is 2 and x+y is 3);

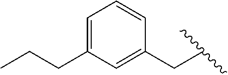 [SMILES: *Cc1cccc(CCC)c1]

(i.e., where y is 1 and x+y is 3); or

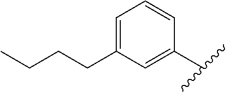 [SMILES: *c1cccc(CCCC)c1]

(i.e., where y is 0 and x+y is 3), and the like.

In cases where the phenyl is substituted with a substituted C₁-C₆ alkyl, non-limiting examples may include a C₁-C₆ alkyl substituted with a phenyl (where the bond to the remaining Formula I molecule is on the far right of the illustrated structures and is shown connected to the rest of Formula I with the wavy bond):

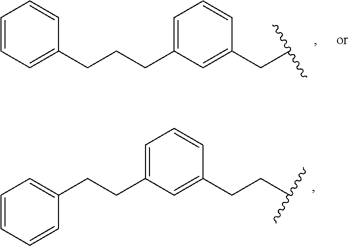 [SMILES: *Cc1cccc(CCCc2ccccc2)c1 ; *CCc1cccc(CCc2ccccc2)c1]

and the like.

In particular, compounds of Formula I with the groups and substituents as set forth below with respect to G₁ may be used for the prevention and/or treatment of cancer:
(CH₂)ₙOH wherein n is 1 or 2, or n is preferably 1;
(CH₂)ₙCH(CH₃)OH wherein n is 1 or 2, or n is preferably 1;
(CH₂)ₙO—CH₃ wherein n is 1 or 2, or n is preferably 1;
(CH₂)ₙ(O)NH₂ wherein n is 1 or 2, or n is preferably 1;
CH₂—COH;
CH(CH₃)OH;
(CH₂)ₙ—C(O)—R₃;
—C(CH₃)₂OH;
—CH(F)—OH;
—CF₂—OH;
—C(O)CH₃
—C(O)—R₃;
—CH₂C(O)OR₃;
—(CH₂)ₙC(O)R₃.

According to a particular embodiment, the compounds are provided as the pharmaceutically acceptable alcohol at position G₁.

According to a particular embodiment, the compounds are provided as the pharmaceutically acceptable aldehyde at position G₁.

According to a particular embodiment, the compounds are provided as the pharmaceutically acceptable ketone at position G₁.

According to a particular embodiment, the compounds are provided as the pharmaceutically acceptable amine at position G₁.

According to a particular embodiment, the compounds are provided as the pharmaceutically acceptable amide at position G₁.

According to a particular embodiment, the compounds are provided as the pharmaceutically acceptable ester at position G₁.

According to a particular embodiment, the compounds are provided as the pharmaceutically acceptable ether at position G₁.

According to a particular embodiment, the compounds are provided as the pharmaceutically acceptable lactone at position G₁.

According to a particular embodiment, the compounds are provided as the pharmaceutically acceptable ketone at position G₁.

Non-limiting examples of compounds of Formula I include alcohol, aldehyde, amine, amide, ester, ether, lactone or ketone (at position G₁) forms of any of the compounds listed in Table 1 hereinafter (shown as the alcohol form), where position G₂ can be preferably H, NH₂, or OH. In a preferred embodiment, the compound is represented by the alcohol, aldehyde, lactone or ketone (at position G₁) form of any one of the following compounds. Where relevant, one or more of the following compounds can be a pharmaceutical salt form (for example a sodium salt thereof):

TABLE 1

| Compound | Structure |
|---|---|
| 1a | 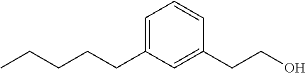 |
| 1b | 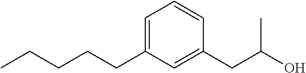 |
| 1c | 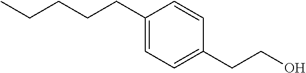 |
| 1d | 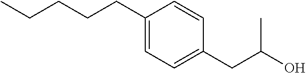 |
| 1e | 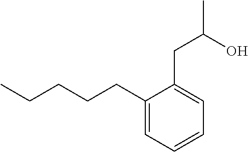 |
| 1f | 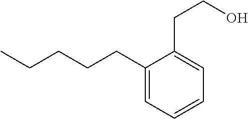 |
| 1g | 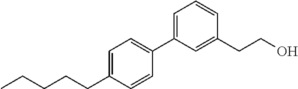 [SMILES: CCCCCc1ccc(-c2cccc(CCO)c2)cc1] |

Non-limiting examples of compounds of Formula I also include alcohol, aldehyde, amine, amide, ester, ether, lactone or ketone (at position G₁) forms of any of the compounds listed in Table 2 hereinafter, where position G₂ can be preferably H, NH₂, or OH. In a preferred embodiment, the compound is represented by the alcohol, lactone, aldehyde or ketone (at position G₁) form of any one of the following compounds. Where relevant, one or more of the following compounds can be a pharmaceutical salt form (for example a sodium salt thereof):

TABLE 2

| Compound | Structure |
|---|---|
| 2a | 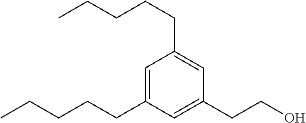 [SMILES: CCCCCc1cc(CCO)cc(CCCCC)c1] |
| 2b | 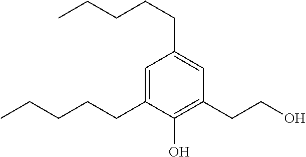 [SMILES: CCCCCc1cc(CCO)c(O)c(CCCCC)c1] |
| 2c | 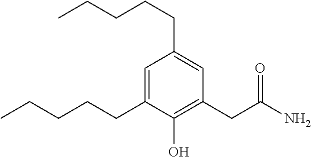 [SMILES: CCCCCc1cc(CCCCC)c(O)c(CC(N)=O)c1] |
| 2d | 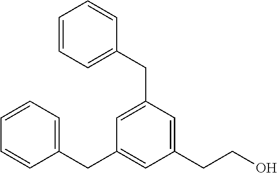 [SMILES: OCCc1cc(Cc2ccccc2)cc(Cc2ccccc2)c1] |

In some embodiments, the compound of Formula I also includes an alcohol, aldehyde, amine, amide, ester, ether or ketone (at position G₁) forms of any of the compounds listed in Table 3 hereinafter, where position G₂ can be preferably H, NH₂, or OH. In a preferred embodiment, the compound is represented by the alcohol, aldehyde, lactone or ketone (at position G₁) form of any one of the following compounds. Where relevant, one or more of the following compounds can be a pharmaceutical salt form (for example a sodium salt thereof):

TABLE 3

| Compound | Structure | Name |
|---|---|---|
| 3a | 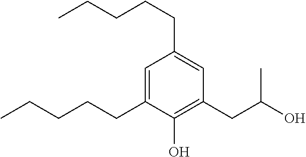 [SMILES: CCCCCc1cc(CCCCC)c(O)c(CC(C)O)c1] | 2-(2-hydroxypropyl)-4,6-dipentylphenol |
| 3b | 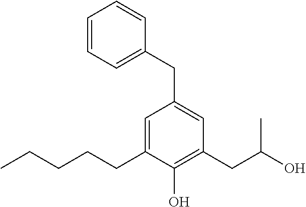 [SMILES: CCCCCc1cc(Cc2ccccc2)cc(CC(C)O)c1O] | 4-benzyl-2-(2-hydroxypropyl)-6-pentylphenol |
| 3c | 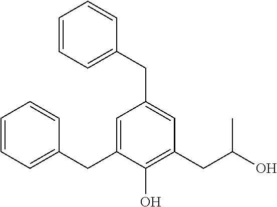 [SMILES: CC(O)Cc1cc(Cc2ccccc2)cc(Cc2ccccc2)c1O] | 2,4-dibenzyl-6-(2-hydroxypropyl)phenol |
| 3d | 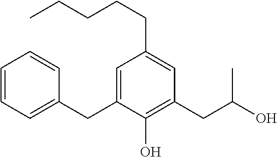 [SMILES: CCCCCc1cc(Cc2ccccc2)c(O)c(CC(C)O)c1] | 2-benzyl-6-(2-hydroxypropyl)-4-pentylphenol |
| 3e | 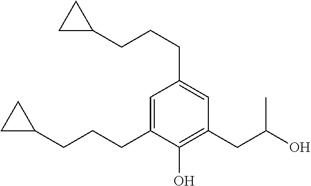 [SMILES: CC(O)Cc1cc(CCCC2CC2)cc(CCCC2CC2)c1O] | 2,4-bis(3-cyclopropylpropyl)-6-(2- hydroxypropyl)phenol |
| 3f | 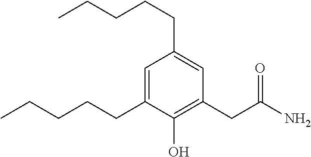 [SMILES: CCCCCc1cc(CCCCC)c(O)c(CC(N)=O)c1] | 2-(2-hydroxy-3,5-dipentylphenyl)acetamide |
| 3g | 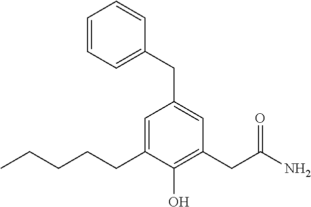 [SMILES: CCCCCc1cc(Cc2ccccc2)cc(CC(N)=O)c1O] | 2-(5-benzyl-2-hydroxy-3- pentylphenyl)acetamide |
| 3h | 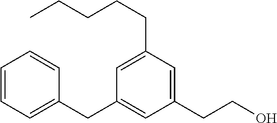 [SMILES: CCCCCc1cc(CCO)cc(Cc2ccccc2)c1] | 2-(3-benzyl-5-pentylphenyl)ethan-1-ol |
| 3i | 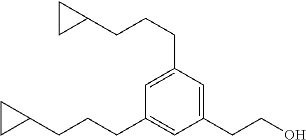 [SMILES: OCCc1cc(CCCC2CC2)cc(CCCC2CC2)c1] | 2-(3,5-bis(3-cyclopropylpropyl)phenyl) ethan-1-o |
| 3j | 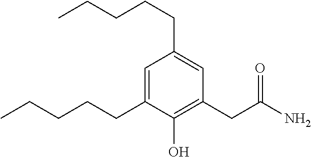 [SMILES: CCCCCc1cc(CCCCC)c(O)c(CC(N)=O)c1] | 2-(2-hydroxy-3,5-dipentylphenyl)acetamide |
| 3k | 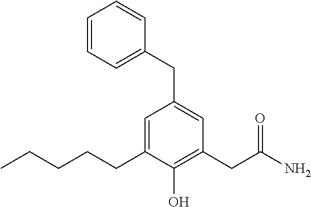 [SMILES: CCCCCc1cc(Cc2ccccc2)cc(CC(N)=O)c1O] | 2-(5-benzyl-2-hydroxy-3- pentylphenyl)acetamide |
| 3l | 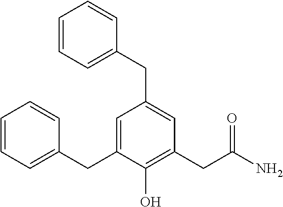 [SMILES: NC(=O)Cc1cc(Cc2ccccc2)cc(Cc2ccccc2)c1O] | 2-(3,5-dibenzyl-2-hydroxyphenyl)acetamide |
| 3m | 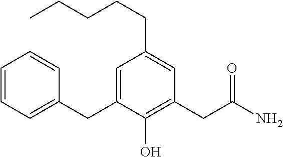 [SMILES: CCCCCc1cc(CC(N)=O)c(O)c(Cc2ccccc2)c1] | 2-(3-benzyl-2-hydroxy-5- pentylphenyl)acetamide |
| 3n | 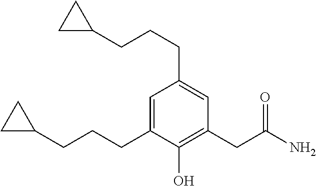 [SMILES: NC(=O)Cc1cc(CCCC2CC2)cc(CCCC2CC2)c1O] | 2-(3,5-bis(3-cyclopropylpropyl)-2- hydroxyphenyl) acetamide |
| 3o | 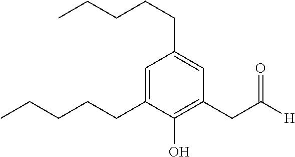 [SMILES: CCCCCc1cc(CC=O)c(O)c(CCCCC)c1] | 2-(2-hydroxy-3,5- dipentylphenyl)acetaldehyde |
| 3p | 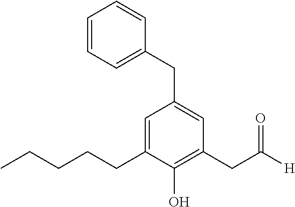 [SMILES: CCCCCc1cc(Cc2ccccc2)cc(CC=O)c1O] | 2-(5-benzyl-2-hydroxy-3- pentylphenyl)acetaldehyde |
| 3q | 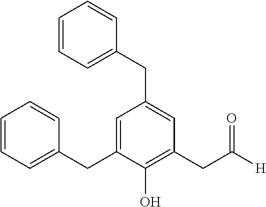 [SMILES: O=CCc1cc(Cc2ccccc2)cc(Cc2ccccc2)c1O] | 2-(3,5-dibenzyl-2- hydroxyphenyl)acetaldehyde |
| 3r | 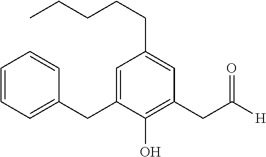 [SMILES: CCCCCc1cc(CC=O)c(O)c(Cc2ccccc2)c1] | 2-(3-benzyl-2-hydroxy-5- pentylphenyl)acetaldehyde |
| 3s | 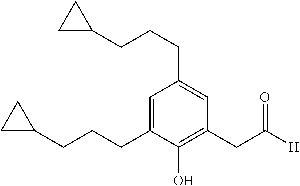 [SMILES: O=CCc1cc(CCCC2CC2)cc(CCCC2CC2)c1O] | 2-(3,5-bis(3-cyclopropylpropyl)-2- hydroxyphenyl)acetaldehyde |
| 3t | 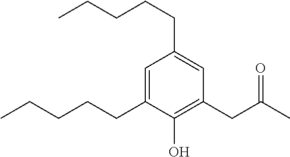 [SMILES: CCCCCc1cc(CCCCC)c(O)c(CC(C)=O)c1] | 1-(2-hydroxy-3,5-dipentylphenyl)propan-2- one |
| 3u | 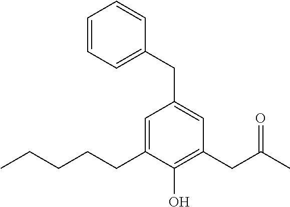 [SMILES: CCCCCc1cc(Cc2ccccc2)cc(CC(C)=O)c1O] | 1-(5-benzyl-2-hydroxy-3- pentylphenyl)propan-2-one |
| 3v | 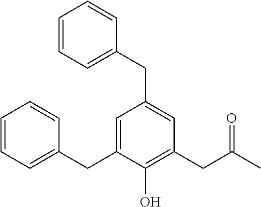 [SMILES: CC(=O)Cc1cc(Cc2ccccc2)cc(Cc2ccccc2)c1O] | 1-(3,5-dibenzyl-2-hydroxyphenyl)propan-2- one |
| 3w | 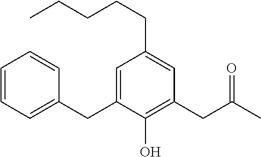 [SMILES: CCCCCc1cc(CC(C)=O)c(O)c(Cc2ccccc2)c1] | 1-(3-benzyl-2-hydroxy-5- pentylphenyl)propan-2-one |
| 3x | 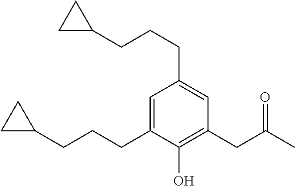 [SMILES: CC(=O)Cc1cc(CCCC2CC2)cc(CCCC2CC2)c1O] | 1-(3,5-bis(3-cyclopropylpropyl)-2- hydroxyphenyl)propan-2-one |
| 3y | 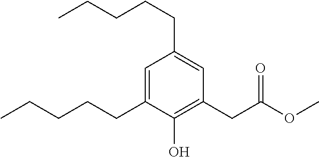 [SMILES: CCCCCc1cc(CCCCC)c(O)c(CC(=O)OC)c1] | methyl 2-(2-hydroxy-3,5- dipentylphenyl)acetate |
| 3z | 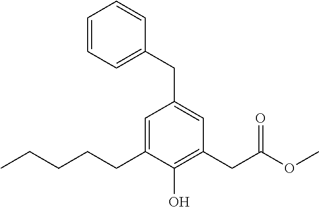 [SMILES: CCCCCc1cc(Cc2ccccc2)cc(CC(=O)OC)c1O] | methyl 2-(5-benzyl-2-hydroxy-3- pentylphenyl)acetate |
| 3aa | 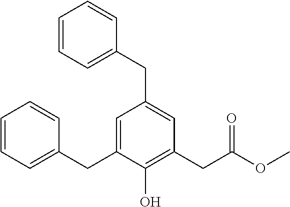 [SMILES: COC(=O)Cc1cc(Cc2ccccc2)cc(Cc2ccccc2)c1O] | methyl 2-(3,5-dibenzyl-2- hydroxyphenyl) acetate |
| 3ab | 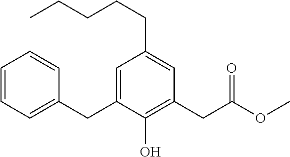 [SMILES: CCCCCc1cc(CC(=O)OC)c(O)c(Cc2ccccc2)c1] | methyl 2-(3-benzyl-2-hydroxy-5- pentylphenyl)acetate |
| 3ac | 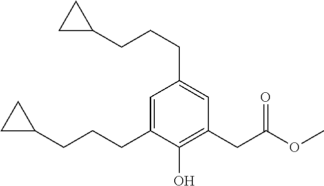 [SMILES: COC(=O)Cc1cc(CCCC2CC2)cc(CCCC2CC2)c1O] | methyl 2-(3,5-bis(3-cyclopropylpropyl)-2- hydroxyphenyl) acetate |
| 3ad | 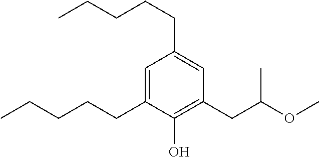 [SMILES: CCCCCc1cc(CCCCC)c(O)c(CC(C)OC)c1] | 2-(2-methoxypropyl)-4,6-dipentylphenol |
| 3ae | 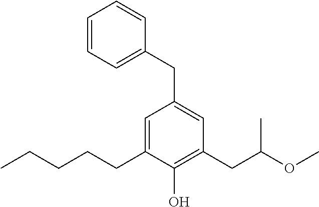 [SMILES: CCCCCc1cc(Cc2ccccc2)cc(CC(C)OC)c1O] | 4-benzyl-2-(2-methoxypropyl)-6- pentylphenol |
| 3af | 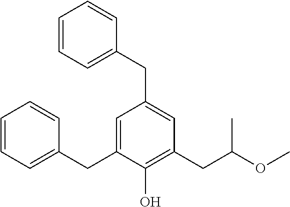 [SMILES: COC(C)Cc1cc(Cc2ccccc2)cc(Cc2ccccc2)c1O] | 2,4-dibenzyl-6-(2-methoxypropyl)phenol |
| 3ag | 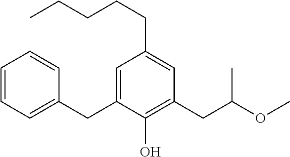 [SMILES: CCCCCc1cc(Cc2ccccc2)c(O)c(CC(C)OC)c1] | 2-benzyl-6-(2-methoxypropyl)-4- pentylphenol |
| 3ah | 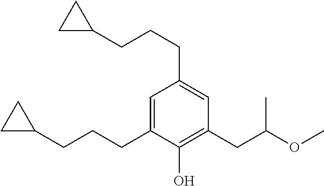 [SMILES: COC(C)Cc1cc(CCCC2CC2)cc(CCCC2CC2)c1O] | 2,4-bis(3-cyclopropylpropyl)-6-(2- methoxypropyl)phenol |
| 3ai | 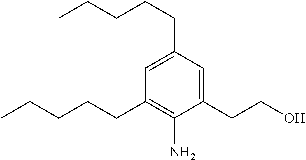 [SMILES: CCCCCc1cc(CCO)c(N)c(CCCCC)c1] | 2-(2-amino-3,5-dipentylphenyl)ethan-1-ol |

### Salts

As used herein, the term “pharmaceutically acceptable salt” is intended to mean base addition salts. Example of pharmaceutically acceptable salts are also described, for example, in Berge et al., “Pharmaceutical Salts”, J. Pharm. Sci. 66, 1-19 (1977). Pharmaceutically acceptable salts may be synthesized from the parent agent that contains an acidic moiety, by conventional chemical methods. Generally, such salts and are prepared by reacting the free acid forms of these agents with a stoichiometric amount of the appropriate base in water or in an organic solvent, or in a mixture of the two. Likewise, when the parent agent contains a group such as —NH₂, the pharmaceutically acceptable salts may be synthesized from the parent agent by conventional chemical methods by reacting the free —NH₃⁺ with an anionic source in a suitable solvent.

Salts may be prepared in situ, during the final isolation or purification of the compound or by separately reacting a purified compound of the present disclosure with the desired corresponding base, and isolating the salt thus formed. For example, this approach may be implemented with the alcohol form of some of the compounds of the present disclosure (such as the alcohol form of at least some of the compounds of any one of Tables 1-3) or with the free acid form of some of the compounds of the present disclosure (such as the free acid form present on a substituent at a position other than G₁ of at least some of the compounds of any one of Tables 1-3).

The pharmaceutically acceptable salt of the compounds of the present disclosure may be selected from the group consisting of organic or inorganic salts.

For example, the pharmaceutically acceptable salt may include a sodium, potassium, calcium, magnesium, lithium, ammonium, manganese, zinc, iron, olamine, meglumine, lysine, tromethamine, or copper salt, when the compounds are amenable to be such salts. In preferred embodiments, the pharmaceutically acceptable salt of the compounds of the present disclosure may be the sodium, potassium, calcium, magnesium or lithium salt, when the compounds are amenable to be such salts. More preferably the pharmaceutically acceptable salt is sodium, when the compounds are amenable to be such salts.

For example, the pharmaceutically acceptable salt may include an acetate, benzoate, besylate, bromide, carbonate, citrate, edisylate, estolate, fumarate, gluconate, hippurate, iodide, maleate, mesylate, methylsulfate, napsylate, oxalate, pamoate, phosphate, stearate, succinate, sulfate, tartrate, tosylate, or chloride salt, when the compounds are amenable to be such salts.

In some embodiments, the compounds are the sodium salts of at least some of the compounds listed in Tables 1-3 hereinbefore, which are amenable to be such salts.

All alcohol, salt and other ionic and non-ionic forms of the compounds described are included when referring to a given compound, where applicable. For example, if a compound is shown as an alcohol herein, the salt forms of the compound are also included, when the compounds are amenable to be such salts. Likewise, if a compound is shown as a salt herein, then the alcohol forms are also included. The same is also applicable to a compound having an aromatic group in one of the substituent groups, where such aromatic group on the substituent group may include a free form of a carboxylic acid. In such case, when the compound is shown as a salt herein, then the carboxylic acid free form is also included. Likewise, when the aromatic group on the substituent group is shown with a free form of a carboxylic acid, then the salt forms of the compound are also included, when the compounds are amenable to be such salts.

### Prodrugs

In certain embodiments, the compounds of the present disclosure may also include all pharmaceutically acceptable salts, isosteric equivalents such as tetrazole and prodrug forms thereof. Examples of the latter include the pharmaceutically acceptable esters or amides of the compounds of the present disclosure.

### Chirality

The compounds of the present disclosure, their pharmaceutically acceptable salts, or prodrugs thereof, may contain one or more asymmetric centers, chiral axes and chiral planes and may thus give rise to enantiomers, diastereomers, and other stereoisomeric forms and may be defined in terms of absolute stereochemistry, such as (R)- or (S)-. The present disclosure is intended to include all such possible isomers, as well as, their racemic and optically pure forms. Optically active (+) and (−), (R)- and (S)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively, specific enantiomers may be synthesized by asymmetric synthesis using optically active reagents, substrates, catalysts, or solvents or by converting one enantiomer to another by asymmetric transformation.

Certain compounds of the present disclosure may exist in Zwitterionic form and the present disclosure includes Zwitterionic forms of these compounds and mixtures thereof.

### Hydrates

In addition, the compounds of the present disclosure also may exist in hydrated and anhydrous forms. Hydrates of any of the formulas described herein may thus exist as a monohydrate or in the form of a polyhydrate.

### B) Methods of Preparation

In general, the compounds of the present disclosure may be prepared by any conventional methods, using readily available and/or conventionally preparable starting materials, reagents and conventional synthesis procedures. Of particular interest is the work of Hundertmark, et al., Org. Lett. 2000, 12, pp. 1729-1731 and WO 2014/138906.

The exemplification section hereinafter provides general schemes and specific, but non limitative, examples for the synthesis of representative compounds of Formula I.

### C) Pharmaceutical Applications

As indicated and exemplified herein, the compounds of the present disclosure may have beneficial pharmaceutical properties and these compounds may have useful pharmaceutical applications in subjects. Medical and pharmaceutical applications contemplated by the inventor include, but are not limited to, prevention and/or treatment of various cancers, oxidative stress associated conditions, inflammatory-related disease, pain, metabolic disorder, and/or fibrosis and fibrosis-related diseases.

In one aspect, the medical and pharmaceutical application is prevention and/or treatment of various cancers. In one embodiment. the cancer is selected from bladder, breast, colorectal, kidney, melanoma, non-Hodgkin's lymphoma, leukemia, ovarian, pancreatic, prostate and uterine cancers.

In another embodiment, the cancer is selected from glioblastoma, heart, esophageal, liver and bile duct, pancreas, lung, gallbladder, mesothelioma, diffuse intrinsic pontine glioma, and acute myelomonocytic leukemia, and fibrosarcoma.

In another embodiment, the cancer is selected from glioblastoma, breast, colorectal, leukemia, melanoma and pancreatic cancers.

In another embodiment, the cancer is selected from brain and skin cancers.

In some embodiments, the pharmaceutical application may include a method of preventing or treating a cancer as defined herein, where the method may include administering to the patient a therapeutically effective amount of the compound of as defined herein, for example, to the proximity of the cancer or in situ at cancer site after removal or not of the primary tumor, in other words peri-tumoral, or pre/post surgical resection of a tumor. In some cases, such administration may occur in the context of inoperable tumors.

In some embodiments, the compound of the present disclosure may be formulated into a slow or controlled release composition, for example for local delivery of the compound at a target site. Slow or controlled release compositions are known in the art (e.g., thermogel) and for conciseness' sake will not be further described here.

Reference herein to treatment extends to prophylaxis as well as therapy of an established cancer. Accordingly, at least some of the compounds of the present disclosure could be used after surgical removal of the primary tumor, prior to surgery, prior or after aggressive chemotherapy, radiotherapy, immunotherapy or other targeted therapy or even when the patient is in remission. These at least some of the compounds of the present disclosure are expected to have a relative lack of toxicity when compared to standard cancer therapies thereby allowing for a more liberal prophylactic use than would be advisable with standard therapies.

In one aspect, the medical and pharmaceutical application is prevention and/or treatment of fibrosis and fibrosis-related diseases. In one embodiment, the compounds of the present disclosure are for use in monotherapy for the treatment of fibrosis and fibrosis-related diseases. For example, the compounds may be used for reducing proliferation or progression of fibrotic tissue in fibrotic-related diseases. In other embodiments, the compounds of the present disclosure are used in combination with one or more already approved anti-fibrosis and anti-fibrosis-related diseases agents such as pirfenidone, nintedanib or other preclinical compounds such as PPAR agonists/antagonists, fezagepras, kinase inhibitors, mTOR inhibitors, and the like.

For example, the fibrotic disease can be pulmonary fibrosis. In this embodiment, the therapeutically effective amount is preferably between about 1 to about 50 mg/kg, and preferably between about 1 to about 20 mg/kg. The compound is preferably administered orally. The subject is preferably a human. According to a preferred embodiment, the pulmonary fibrosis is idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension.

For example, the fibrotic disease can be liver fibrosis. In this embodiment, the therapeutically effective amount is preferably between about 1 to about 50 mg/kg. The compound is preferably administered orally. The subject is preferably human. According to a preferred embodiment, the liver fibrosis is resulting from a chronic liver disease, hepatitis B virus infection, hepatitis C virus infection, hepatitis D virus infection, schistosomiasis, alcoholic liver disease or non-alcoholic steatohepatitis, obesity, diabetes, protein malnutrition, coronary artery disease, auto-immune hepatitis, cystic fibrosis, alpha-1-antitrypsin deficiency, primary biliary cirrhosis, drug reaction and exposure to toxins.

For example, the fibrotic disease can be skin fibrosis. In this embodiment, the compound is preferably administered topically or orally. When administered topically, the therapeutically effective amount of the compound of the present disclosure is preferably between about 0.01 to about 10% (w/w). The subject is preferably human. When administered orally, the therapeutically effective amount of the compound of the present disclosure is preferably between about 1 to about 50 mg/kg and the subject is human. According to a preferred embodiment of the disclosure, the skin fibrosis is scarring, hypertrophic scarring, keloid scarring, dermal fibrotic disorder, wound healing, delayed wound healing, psoriasis or scleroderma. Said scarring may derived from a burn, a trauma, a surgical injury, a radiation or an ulcer. Said ulcer can be a diabetic foot ulcer, a venous leg ulcer or a pressure ulcer.

For example, the fibrotic disease can be cardiac fibrosis. In this embodiment, the therapeutically effective amount is preferably between about 1 to about 50 mg/kg, and preferably between about 1 to about 20 mg/kg. The compound is preferably administered orally. The subject is preferably a human. According to a preferred embodiment, the cardiac fibrosis is resulting from coronary artery and vascular diseases, myocardial infarction, heart failure, atherosclerosis, angina, arrhythmia.

For example, the fibrotic disease can be renal fibrosis. In this embodiment, the therapeutically effective amount is preferably between about 1 to about 50 mg/kg, and preferably between about 1 to about 20 mg/kg. The compound is preferably administered orally. The subject is preferably a human. According to a preferred embodiment, the renal fibrosis may result from chronic kidney diseases (CKD), acute kidney diseases (AKD), diabetic kidney diseases (DKD), polycystic kidney diseases (PKD), or other rare or genetic diseases.

In addition to the previous embodiments of dosages, for all above mentioned fibrotic diseases, when the compound of the present disclosure is topically administered to a human, the therapeutically effective amount of a compound corresponds to preferably between about 0.01 to about 10% (w/w), or between about 0.1 to 10% (w/w), or between about 1.0 to about 10% (w/w), between about 0.1 to about 5% (w/w), or between about 1.0 to about 5% (w/w). In all above-mentioned fibrotic diseases, when the compound of the present disclosure is orally administered to a human, the therapeutically effective amount of a compound corresponds preferably between about 1 to about 50 mg/kg, or between about 1 to 25 mg/kg, or between about 1 to about 10 mg/kg, between about 5 to about 25 mg/kg, or between about 10 to about 20 mg/kg.

In one aspect, the medical and pharmaceutical application is prevention and/or treatment of an oxidative stress related disorder. The term “oxidative stress related disorder” refers to any disease in which there is an imbalance between the production of reactive oxygen species and a biological system's ability to readily detoxify the reactive intermediates or easily repair the resulting damage. Examples of such diseases include, but are not limited to, cardiovascular diseases, cancer, diabetes, arthritis, atherosclerosis, Parkinson's disease, heart failure, myocardial infarction, Alzheimer's disease, chronic fatigue syndrome and autoimmune diseases.

In one aspect, the medical and pharmaceutical application is prevention and/or treatment of inflammatory-related diseases. The term “inflammatory-related disease” refers to any and all abnormalities associated with inflammatory-related disease, including chronic and acute inflammatory diseases, including but not limited to immune mediated inflammatory diseases (IMID) and autoimmune diseases arthritis, ITP, glomerulonephritis, vasculitis, psoriatic arthritis, systemic lupus erythematosus (SLE), idiopathic thrombocytopenic purpura (ITP), psoriasis, Crohn's disease, inflammatory bowel disease, ankylosing spondylitis, Sjogren's syndrome, Still's disease (macrophage activation syndrome), uveitis, scleroderma, myositis, Reiter's syndrome, and Wegener's syndrome. For example, the inflammatory-related disease may include rheumatoid arthritis, oedema, dermatitis, colitis and others. In general, prophylactic and therapeutic uses comprise the administration of a compound as described herein to a subject, preferably a human patient in need thereof. The compounds of the present disclosure may be administered with any conventional treatments. In order to evaluate, assess, and/or confirm the efficacy of the method, compounds and/or compositions of the disclosure, serial measurements can be determined. Quantitative methods and techniques for the assessment of inflammatory-related disease are well known in the art.

In one aspect, the medical and pharmaceutical application is prevention and/or treatment of metabolic diseases or disorders. Some of the symptoms that can occur with metabolic disorders are lethargy, weight loss, jaundice and seizures. It is believed that the principal classes of metabolic disorders are: acid-base imbalance, metabolic brain diseases, disorders of calcium metabolism, DNA repair-deficiency disorders, glucose metabolism disorders, hyperlactatemia, iron metabolism disorders, lipid metabolism disorders, malabsorption syndromes, metabolic syndrome X, inborn error of metabolism, mitochondrial diseases, phosphorus metabolism disorders, porphyrias, proteostasis deficiencies, metabolic skin diseases, wasting syndrome, or water-electrolyte imbalance. For example, the metabolic diseases or disorders may include diabetes type 1, II or Ill, triglyceridemia, cholesterolemia, and others.

In some embodiments, the compounds of the present disclosure pharmaceutically acceptable salt thereof are for use in monotherapy for the treatment of cancer.

In other embodiments, the compound of the present disclosure or pharmaceutically acceptable salt thereof is used in combination with one or more already approved anticancer therapy, such as but without being limited to chemotherapeutic agents, cytokines, radiation therapy agents, immunotherapy, monoclonal antibodies, targeted therapy, etc. Examples of anticancer agents which may be used in combination with the compounds of the present disclosure include, but are not limited to, temozolomide, abraxane, decarbazine, doxorubicin, daunorubicin, cyclophosphamide, busulfex, busulfan, bleomycin, alectinib, melphalan, pamidronate, bevacizumab, carbozantinib, vinblastine, docetaxel, prednisolone, ifosphamide, dexamethasone, vincristine, bleomycin, etoposide, topotecan, mitomycine, irinotecan, taxotere, taxol, 5-fluorouracil, folfirinox, methotrexate, gemcitabine, cisplatin, carboplatin, chlorambucil, beribucin and tyrosine kinase inhibitors.

In some embodiments, a method of treatment or prevention according to the present disclosure may also include co-administration of the at least one compound according to the present disclosure, or a pharmaceutically acceptable salt thereof together with the administration of another therapeutically effective agent. Therefore, an additional aspect of the present disclosure relates to methods of concomitant therapeutic treatment of a subject, comprising administering to a subject in need thereof an effective amount of a first agent and a second agent, wherein the first agent is as defined in Formula I or other listed compounds, and the second agent is for the prevention or treatment of any one of disorder or disease as defined hereinbefore. As used herein, the term “concomitant” or “concomitantly” as in the phrases “concomitant therapeutic treatment” or “concomitantly with” includes administering a first agent in the presence of a second agent. A concomitant therapeutic treatment method includes methods in which the first, second, third or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed stepwise by different actors. For example, one actor may administer to a subject a first agent and as a second actor may administer to the subject a second agent and the administering steps may be executed at the same time, or nearly the same time, or at distant times, so long as the first agent (and/or additional agents) are after administration in the presence of the second agent (and/or additional agents). The actor and the subject may be the same entity (e.g., a human).

Accordingly, the present disclosure also relates to a method for preventing, reducing or eliminating a symptom or complication or metastasis of any one of the above-mentioned diseases or conditions. The method comprises administering, to a subject in need thereof, a first pharmaceutical composition comprising at least one compound of the present disclosure and a second pharmaceutical composition comprising one or more additional active ingredients, wherein all active ingredients are administered in an amount sufficient to inhibit, reduce, or eliminate one or more symptoms or complications of the disease or condition to be treated. In one aspect, the administration of the first and second pharmaceutical composition is temporally spaced apart by at least about two minutes. Preferably the first agent is a compound of Formula I or other listed compounds as defined herein, or a pharmaceutically acceptable salt thereof, e.g., sodium salt, when the compounds are amenable to be such salts. The second agent may be selected from the list of compounds given hereinbefore but not limited thereto.

### D) Pharmaceutical Compositions and Formulations

As indicated hereinbefore, at least some of the compounds of the present disclosure may have one or more potential therapeutic applications. Therefore, the disclosure is also concerned with pharmaceutical compositions comprising a therapeutically effective amount of one or more of the compounds of the present disclosure and a pharmaceutically acceptable carrier, diluent or excipient. For example, such pharmaceutical compositions may include a compound of Formula I, such as the alcohol, aldehyde, ester, ketone forms of those compounds described in any one of the aforementioned Tables.

A related aspect of the present disclosure concerns pharmaceutical compositions comprising a therapeutically effective amount of one or more of the compounds of the present disclosure. As indicated hereinbefore, the pharmaceutical compositions of the present disclosure may be useful in prevention and/or treatment of one or more cancers in subjects.

The composition of the present disclosure may include one or more compounds of Formula I or other listed compounds, as defined herein or pharmaceutically acceptable derivatives, salts prodrugs, analogues and isomers, or enantiomers thereof. Formulations of the active compound may be prepared so as to provide a pharmaceutical composition in a form suitable for enteral (such as taken orally in the form of liquids, capsules, tablets, or chewable tablets), mucosal (including sublingual, nasally, breathed into the lungs, usually through the mouth (by inhalation) or mouth and nose (by nebulization), vaginal and rectal), parenteral (including intramuscular, intradermal, intrathecally, subcutaneous and intravenous) or topical (including ointments, creams or lotions) administration. The formulation may, where appropriate, be conveniently presented in discrete dosage units and may be prepared by any of the methods well-known in the art of pharmaceutical formulation. All methods include the step of bringing together the active pharmaceutical ingredient with liquid carriers or finely divided solid carriers or both as the need dictates.

When appropriate, the above-described formulations may be adapted to provide sustained release of the active pharmaceutical ingredient. Sustained release formulations well-known to the art include the use of a bolus injection, continuous infusion, biocompatible polymers, chitosan or liposomes. The herein described compound(s) may also be administered in situ at the site of the primary cancer, as depot.

### E) Kits

The compound(s) of the present disclosure may be packaged as part of a kit, optionally including a container (e.g., packaging, a box, a vial, etc.). The kit may be commercially used according to the methods described herein and may include instructions for use in a method of the present disclosure. Additional kit components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The compound(s) of the present disclosure may or may not be administered to a patient at the same time or by the same route of administration. Therefore, the methods of the present disclosure encompass kits which, when used by the medical practitioner, can simplify the administration of appropriate amounts of two or more active ingredients to a patient.

A typical kit of the present disclosure comprises a unit dosage form of at least one compound according to the disclosure as defined by Formula I, or a pharmaceutically acceptable salt thereof, and a unit dosage form of at least one additional active ingredient. Examples of additional active ingredients that may be used in conjunction with the compounds of the present disclosure include, but are not limited to, any of the anticancer agents indicated hereinbefore that could be used in combination with the compound(s) of the present disclosure. The kit may further include instructions for the use as described herein, on a suitable media such as but without being limited to a paper insert, a computer readable media, and the like.

Kits of the present disclosure can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles are provided herein before.

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

As used herein, the term “therapeutically effective amount” means the amount of compound that, when administered to a subject for treating or preventing a particular disorder, disease or condition, is sufficient to effect such treatment or prevention of that disorder, disease or condition. As used herein, the term “therapeutically effective amount” further means the amount of compound that induces regression of established tumors and/or primary solid tumors; inhibits cell proliferation, cancer cell migration, and metastasis. Dosages and therapeutically effective amounts may vary for example, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject (e.g., total or partial response as evidenced by factors which include reduction in tumor burden and/or tumor size as well as increase in survival time and/or quality of life which is associated with a reduction in amount and/or duration of treatment with standard but more toxic anticancer agents), the properties of the compounds (e.g., bioavailability, stability, potency, toxicity, etc.), and the particular disorder(s) the subject is suffering from. In addition, the therapeutically effective amount may depend on the subject's blood parameters (e.g., lipid profile, insulin levels, glycemia), the severity of the disease state, organ function, or underlying disease or complications. Such appropriate doses may be determined using any available assays including the ex-ovo (chorioallantoic membrane) assays described herein. When one or more of the compounds of the present disclosure is to be administered to humans, a physician may for example, prescribe a relatively low dose at first, subsequently increasing the dose until an appropriate response is obtained. The dose to be administered will ultimately be at the discretion of the oncologist. In general, however, the dose will be in the range from about 1 to about 100 mg/kg per day when administered orally; and in the range from about 0.01 to about 10 mg/kg per day when administered intravenously or subcutaneously.

As used herein, the term “pharmaceutically acceptable carrier”, “pharmaceutically acceptable diluent or “pharmaceutically acceptable excipient” is intended to mean, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or encapsulating agent, such as a liposome, sodium decanoate, triglyceride, cyclodextrins, encapsulating polymeric delivery systems or polyethyleneglycol matrix, which is acceptable for use in subjects, preferably humans. It preferably refers to a compound or composition that is approved or approvable by a regulatory agency of the Federal or State government or listed in the U.S. Pharmacopoeia or other generally recognized pharmacopoeia for use in animals and more particularly in humans. The pharmaceutically acceptable vehicle can be a solvent or dispersion medium containing, for example, water, ethanol, polyol (for example, glycerol, propylene glycol, and liquid polyethylene glycol), suitable mixtures thereof, and vegetable oils. Additional examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Prevention of the action of microorganisms can be achieved by addition of antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of injectable compositions can be brought about by including in the composition an agent which delays absorption, for example, aluminum monostearate, chitosan or gelatin.

As used herein, the terms “treatment” or “treating” of a subject relates to the application or administration of a compound of the present disclosure to a subject (or application or administration of a compound of the present disclosure to a cell or tissue from a subject) with the purpose of delaying, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being. In some embodiments, the term “treating” can include increasing a subject's life expectancy and/or delay before additional treatments are required (e.g., dialysis or kidney transplantation for a patient having kidney cancer).

As used herein, the term “preventing” or “prevention” is intended to refer to at least the reduction of likelihood of the risk of (or susceptibility to) acquiring a disease or disorder or metastasis (i.e., causing at least one of the clinical symptoms of the disease not to develop in a patient that may be exposed to or predisposed to the disease but does not yet experience or display symptoms of the disease). Biological and physiological parameters for identifying such patients are provided herein and are also well known by physicians.

As used herein, the term “subject” includes living organisms in which cancers can occur, or which are susceptible to such disease. The term “subject” includes animals such as mammals or birds. Preferably, the subject is a mammal. More preferably, the subject is a human. Most preferably, the subject is a human patient in need of treatment.

As used herein, the term “alkyl” is intended to include both branched and straight chain saturated aliphatic hydrocarbon groups having the specified number of carbon atoms in a linear or branched arrangement, for example, C₁-C₈ as in C₁-C₈ alkyl is defined as including groups having 1, 2, 3, 4, 5, 6, 7 or 8 in a linear or branched arrangement. Examples of C₁-C₈ alkyl include, but are not limited to, methyl, ethyl, n-propyl, i-propyl, n-butyl, t-butyl, i-butyl, pentyl, hexyl, heptyl and octyl. In preferred embodiments, the alkyl groups are linear alkyl groups.

As used herein, the term, “alkenyl” is intended to mean unsaturated straight or branched chain hydrocarbon groups having the specified number of carbon atoms therein, and in which at least two of the carbon atoms are bonded to each other by a double bond, and having either E or Z regiochemistry and combinations thereof. For example, C₂-C₆ as in C₂-C₆ alkenyl is defined as including groups having 2, 3, 4, 5, or 6 carbons in a linear or branched arrangement, at least two of the carbon atoms being bonded together by a double bond. Examples of C₂-C₆ alkenyl include ethenyl (vinyl), 1-propenyl, 2-propenyl, and 1-butenyl. In preferred embodiments, the alkenyl groups are linear alkenyl groups.

As used herein, the term “optionally substituted” refers to groups substituted with from 1 to 5 substituents selected from the group consisting of C₁-C₆ alkyl, C₂-C₆ alkenyl, C₂-C₆ alkynyl, C₃-C₈-cycloalkyl, C₃-C₈ heterocycloalkyl, C₁-C₆ alkyl aryl, C₁-C₆ alkyl heteroaryl, C₁-C₆ alkyl cycloalkyl, C₁-C₆ alkyl C₃-C₈heterocycloalkyl, amino, aminosulfonyl, ammonium, acyl amino, amino carbonyl, aryl, heteroaryl, sulfinyl, sulfonyl, alkoxy, alkoxy carbonyl, carbamate, sulfanyl, halogen, trihalomethyl, cyano, hydroxy, mercapto and nitro. Preferably, to groups substituted with from 1 to 5 substituents selected from the group consisting of C₁-C₆ alkyl, C₂-C₆ alkenyl, C₂-C₆ alkynyl, C₃-C₃-cycloalkyl, C₃-C₈ heterocycloalkyl, C₁-C₆ alkyl aryl, C₁-C₆ alkyl heteroaryl, C₁-C₆ alkyl cycloalkyl, and C₁-C₆ alkyl C₃-C₈ heterocycloalkyl.

As used herein, the term “subject” denotes a non-human mammal or a human. Preferred non-human mammals include primates, rodents, such as mouse or rat, feline, canine, bovine and ovine. More particularly, the subject is a human, in particular a child, an adult, a woman or a man.

As used herein, the term “lactones” refers to cyclic carboxylic esters, containing a 1-oxacycloalkan-2-one structure (—C(O)—O—), or analogues having unsaturation or heteroatoms replacing one or more carbon atoms of the ring. Lactones are usually formed by intramolecular esterification of the corresponding hydroxycarboxylic acids, which takes place spontaneously when the ring that is formed is five- or six-membered.

### EXAMPLES

The following examples describe some exemplary modes of making and practicing certain compositions that are described herein. These examples are for illustrative purposes only and are not meant to limit the scope of the compositions and methods described herein.

The examples set forth herein below provide exemplary methods for the preparation of certain representative compounds encompassed by Formula I. Some Examples provide exemplary uses of certain representative compounds of Formula I. Also provided are exemplary methods for assaying representative compounds of Formula I for in vitro, ex ovo and/or in vivo efficacy.

In these examples, the following compounds will be referred in terms of “Representative Compound x” where x is a number from 1 to 5 as per the following

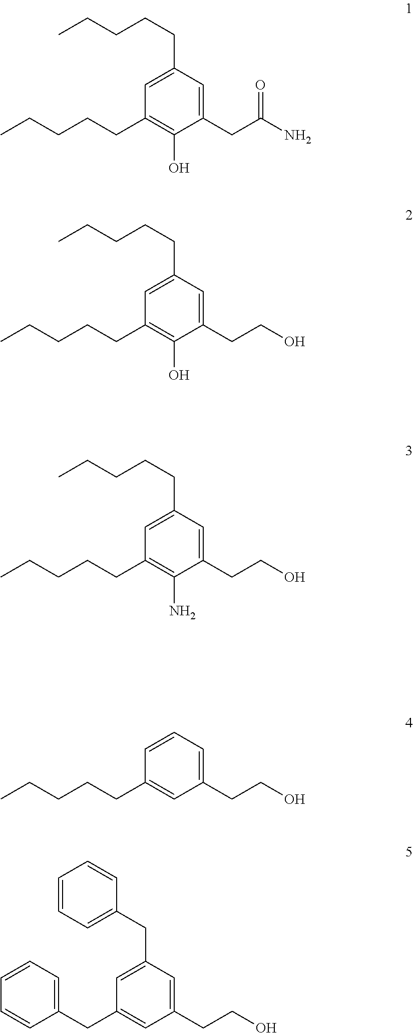 [SMILES: CCCCCc1cc(CCCCC)c(O)c(CC(N)=O)c1 ; CCCCCc1cc(CCO)c(O)c(CCCCC)c1 ; CCCCCc1cc(CCO)c(N)c(CCCCC)c1 ; CCCCCc1cccc(CCO)c1 ; OCCc1cc(Cc2ccccc2)cc(Cc2ccccc2)c1]

### Instrumentation:

All HPLC chromatograms and mass spectra were recorded on an HP 1100 LC-MS Agilent instrument using an analytical C18 column (250×4.6 mm, 5 microns) with a gradient over 3 min of 50-99% CH₃CN—H₂O with 0.01% TFA as the eluant followed by isocratic over 3 min and a flow of 2 mL/min.

### Example 1: Experimental Procedures for the Preparation of Certain Representative Compounds

### A) Representative Compound 2

The following procedure was used to prepare Representative Compound 2:

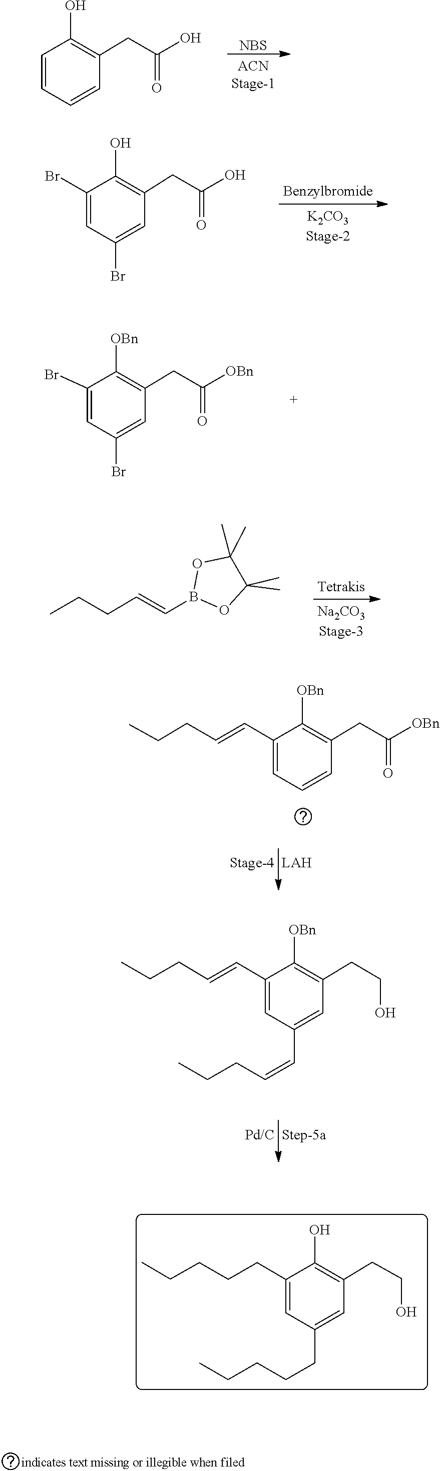

Step-1: To a solution of (2-Hydroxy-phenyl)-acetic acid (1 equiv.), in ACN was added NBS lotwise (2.2 equiv.) at 0° C., then slowly brought the RM for room temperature (rt) then stirred for 16 h at room temperature (rt) (TLC control). The RM was concentrated to remove ACN then digested in Water for 1 h, then filtered and dried the collected solid. The solid was taken in Toluene heated up to 100° C. to dissolve then slowly brought to rt and filtered to get pure compound.

Step-2: To a solution from Step-1 (1 equiv.) in DMF (5 V), was added K₂CO₃ (5 equiv.) and Benzylbromide (2.2 equiv.) dropwise and stirred the RM for 4 h at 80° C. (TLC control). The RM was quenched into water and extracted with Methyl tert-butyl ether (MTBE), washed the MTBE layer with water and dried then concentrated under reduced pressure to get crude.

Step-3: To a solution from Step-2 (1 equiv.), Olefinic Boronic ester derivative (2.2 equiv.) and Na₂CO₃ (5 equiv.) in DME (10 V) and H₂O (1 V) was degassed with argon then added Pd(PPh₃)₄ (0.1 equiv.), then stirred for 18 h at 90° C. Work-Up: The reaction mixture was filtered through Celite then filtrate was diluted with EtOAc and washed with Water, dried the organic layer then concentrated to get crude as dark brown viscous oil. The crude was purified through CombiFlash™ to get pure compound.

Step-4: To a solution from Step-3 (1 equiv.) in Dry THF (10 V) was cooled to 0° C. then added LAH (1.2 equiv.) dropwise under argon atmosphere then brought to rt and stirred for 1 h at rt. The RM was quenched with aq·Na₂SO₄ sol at 0° C. then extracted with EtOAc, dried the organic layer and concentrated under reduced pressure to get crude. The crude was purified through column chromatography.

Step-5: To a solution from Step-4 (1 equiv.) in EtOAc (10 V) was added Pd(OH)₂ (50% w/w) then stirred at rt in autoclave under 10 bar H₂ pressure (TLC control). The RM was filtered through Celite and the filtrate was concentrated to get crude. This crude was subjected to reverse phase CombiFlash purification to obtain the desired product in high purity Purification condition: SiliaSep™ C18, 80 g cartridge was used, with eluent: Gradient elution of MeCN in water (0.1% HCO₂H). Purity was assessed by LCMS and identity confirmed with ¹H nuclear magnetic resonance (¹H NMR).

### B) Representative Compound 1

The following procedure was used to prepare Representative Compound 1:

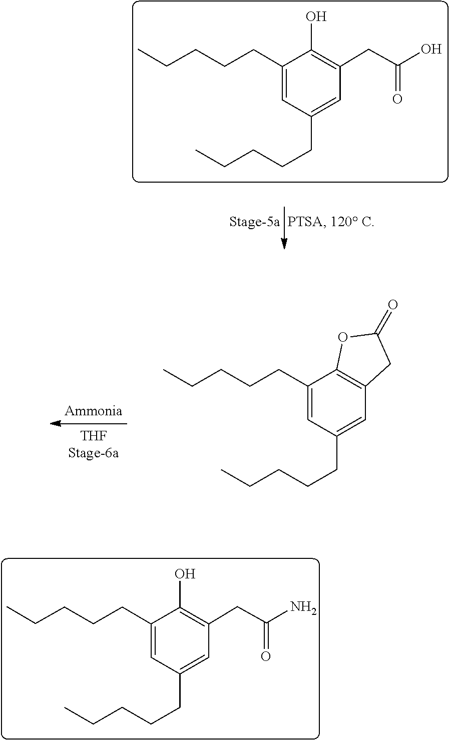 [SMILES: CCCCCc1cc(CCCCC)c(O)c(CC(=O)O)c1 ; CCCCCc1cc(CCCCC)c2c(c1)CC(=O)O2 ; CCCCCc1cc(CCCCC)c(O)c(CC(N)=O)c1]

Step 5a: To a solution of Representative Compound 2 (1 equiv.) in Toluene (20 V), was added p-Toluenesulfonic acid (p-TSA) (0.1 equiv.), then heated the RM to 120-130° C. for 16 h using Dean-Stark condenser. The RM was concentrated to get crude. The crude was purified through column chromatography.

Step-6a: To a solution from Step-5a (1 equiv.) in THF (10 V) was cooled to −20° C., then purged in ammonia gas for 10 min and stirred for 16 h at 80° C. (TLC control). The RM was concentrated to get crude compound. Crude weight−225 mg; purity by LCMS−81%. The crude was recrystallized using hexanes to get the desired compound

### C) Representative Compound 3

The following procedure was used to prepare Representative Compound 3.

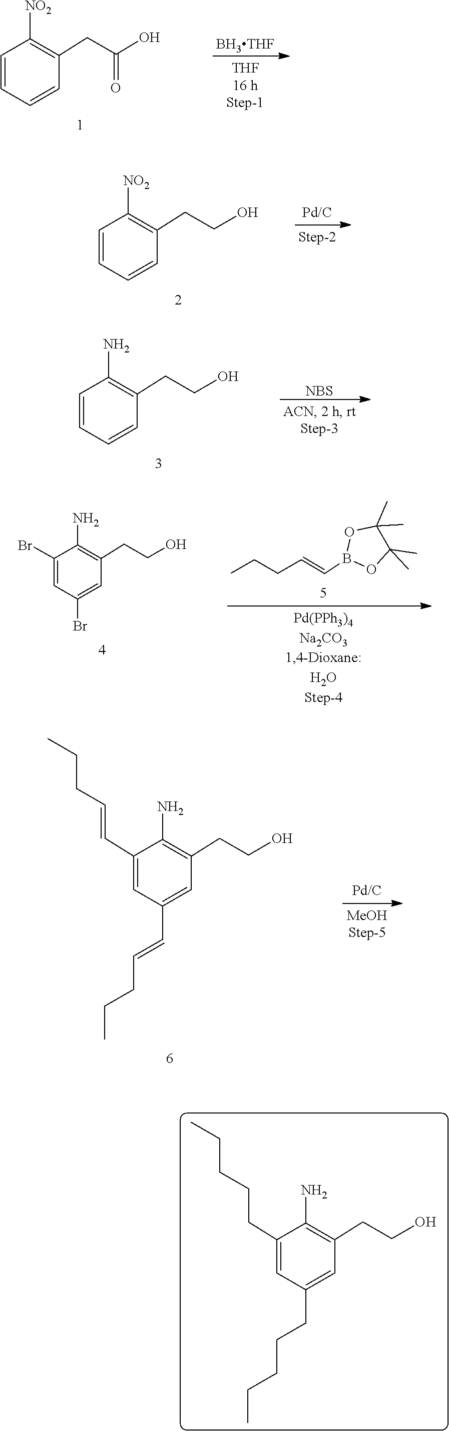

Step-1: To a solution of Int-1 (1 equiv.) in dry THF (10 V) was cooled to 0° C. then added BH₃. THF (1.5 equiv.) dropwise under argon atmosphere then brought to rt and stirred for 16 h at room temperature (rt). The RM was quenched with water at 0° C. then diluted with EtOAc and washed with water, dried the organic layer and concentrated under reduced pressure to get crude. The crude was purified through CombiFlash to get pure compound.

Step-2: To a solution from Step-1 (1 equiv.) in MeOH (10 V) was added Pd/C (20% w/w) then stirred for 16 h at rt under 5 bar H₂ (TLC control). Work-Up: The RM was filtered through Celite and the filtrate was concentrated under reduced pressure to get crude. The crude was purified through column chromatography.

Step-3: To a solution from Step-2 (1 equiv.) in THF (10 V), was added NBS (2.5 equiv.) at 0° C. then stirred for 16 h at rt under (TLC control). Work-Up: The RM was quenched in to sat sodium thiosulfate and extracted with EtOAc, dried the organic layer and concentrated under reduced pressure to get crude. The crude was purified through column chromatography.

Step-4: To a solution from Step-3 (1 equiv.), Olefinic Boronic ester derivative (2.2 equiv.), and Na₂CO₃ (5 equiv.) in 1,4-Dioxane (10 V) and H₂O (1 V) was degassed with argon then added Pd(PPh₃)₄ (0.2 equiv.), then stirred for 18 h at 90° C. Work-Up: The reaction mixture was filtered through Celite then filtrate was diluted with EtOAc and washed with Water, dried the organic layer then concentrated to get crude as dark brown viscous oil. The crude was purified through column chromatography to get pure compound.

Step-5: To a solution from Step-4 (1 equiv.) in MeOH (10 V) was added Pd(OH)₂ (20% w/w), then stirred for 16 h at rt under 5 bar H₂ (TLC control). Work-Up: The RM was filtered through Celite and the filtrate was concentrated under reduced pressure to get crude. The crude was purified through column chromatography to get pure compound. Purity was assessed by LCMS and identity confirmed with ¹H nuclear magnetic resonance (¹H NMR).

### D) Representative Compound 4

The following procedure was used to prepare Representative Compound 4

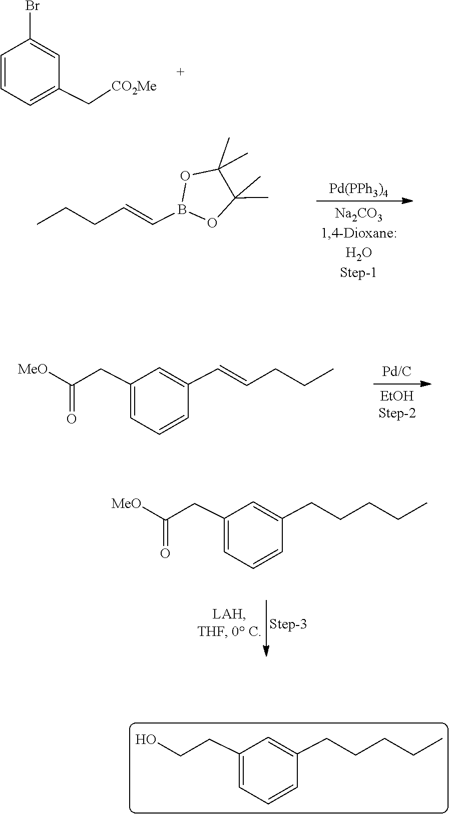 [SMILES: COC(=O)Cc1cccc(Br)c1 ; CCC/C=C/B1OC(C)(C)C(C)(C)O1 ; CCC/C=C/c1cccc(CC(=O)OC)c1 ; CCCCCc1cccc(CC(=O)OC)c1 ; CCCCCc1cccc(CCO)c1 ; "Pd(PPh3)4"]

Step-1: To a solution of (3-Bromophenyl)acetic acid methylester (1 equiv.), Olefinic Boronic ester derivative (1.1 equiv.), and Na₂CO₃ (3 equiv.), in 1,4-Dioxane (10 V), and H₂O (1 V), was degassed with Argon for 15 min then added Pd(PPh₃)₄ (0.01 equiv.), then stirred for 18 h at 90° C. Work-Up: The reaction mixture was filtered through Celite then filtrate was diluted with EtOAc and washed with Water, dried the organic layer then concentrated to get crude as dark brown viscous oil.

Step-2: To a solution from Step-1 (1 equiv.) in EtOH (20 V), was added Pd(OH)₂ (20% w/w) then stirred at rt in autoclave under 5 bar H₂ pressure (TLC control). The RM was filtered through Celite and the filtrate was concentrated to get crude. The crude was purified through combi-flash to get pure compound

Step-3: To a solution from Step-2 (1 equiv.) in dry THF (10 V), was cooled to 0° C. then added lithium aluminum hydride (LAH) (1.2 equiv.) dropwise under argon atmosphere then brought to rt and stirred for 1 h at rt. The RM was quenched with aq. Na₂SO₄ sol at 0° C. then diluted with EtOAc then filtered through celite the filtrate layers, dried the organic layer and concentrated under reduced pressure to get crude. The crude was purified through CombiFlash to get pure compound. Purity was assessed by LCMS and identity confirmed with ¹H nuclear magnetic resonance (¹H NMR).

### E) Representative Compound 5

The following procedure was used to prepare Representative Compound 5.

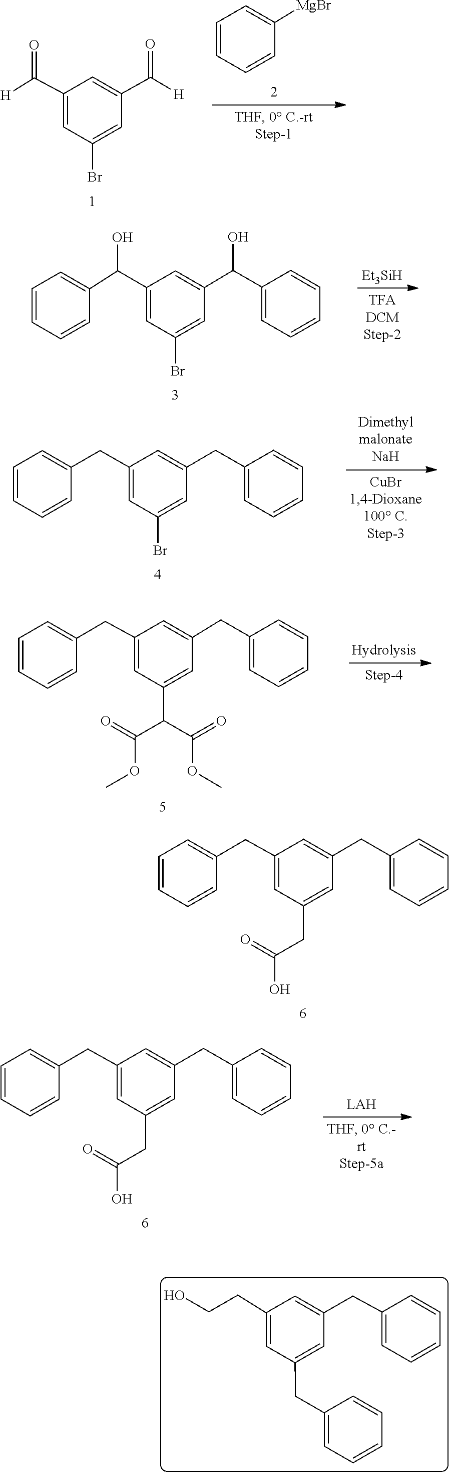

Step-1: To a solution of Int-1 (1 equiv.) in THF (10 V) cooled to 0° C. was added Phenyl magnesium bromide (5 equiv.) (2.0 M in THF) dropwise then stirred for 16 h at rt. (TLC control). Work-Up: The RM was quenched with sat NH4Cl sol then extracted with EtOAc, dried the organic layer and concentrated under reduced pressure to get crude.

Step-2: To a solution from Step-1 (1 equiv.) in dichloromethane (DCM) (10 V) cooled to 0° C. was added Triethylsilane (2.5 V) and Trifluoroacetic acid (TFA) (5 V), dropwise then stirred for 20 h at rt. (TLC control). The RM was diluted with DCM and washed with water, dried the organic layer and concentrated under reduced pressure to get crude. The crude was purified through column chromatography to get pure product.

Step-3: To a solution from Step-2 (1 equiv.), CuBr₂ (2 equiv.) and Dimethylmalonate (2.2 equiv.) in 1,4-Dioxane (10 V), cooled to 0° C. then added NaH (2.0 equiv.) and stirred for 16 h at 100° C. (TLC control). Work-Up: The RM was filtered through Celite and washed the bed with EtOAc the filtrate was concentrated and purified by column chromatography to get pure product.

Step-4: To a solution from Step-3 (1 equiv.) in ethanol (10 V), was added NaOH (2.2 equiv.), at rt then stirred for 16 h at 60° C. (TLC control). Work Up: The RM was diluted with water and washed with MTBE then the aqueous layer was acidified with 1.5 N HCl then extracted with EtOAc, washed the organic layer with water then dried and concentrated under reduced pressure.

Step-5a: To a solution from Step-4 (1 equiv.) in THF (10 V) cooled to 0° C. was added LAH (5 equiv.) (2.0 M in THF) dropwise then stirred for 2 h at rt. (TLC control). Work-Up: The RM was quenched with water then extracted with EtOAc, dried the organic layer and concentrated under reduced pressure to get crude. Purity was assessed by LCMS and identity confirmed with ¹H nuclear magnetic resonance (¹H NMR).

### Example 2: Antitumor Effect of Compounds on Avatar CAM Assay

In this example, representative compounds of the present disclosure were tested for their respective effect on tumors using a model of Avatar Chick Chorioallantoic Membrane (CAM) assay. CAM assays have been widely used to study angiogenesis and tumor invasion of colorectal, prostate and brain cancers, as well as for studying patient-derived xenograft for potential personalized medicine.

The present inventor has tested representative compounds from the present disclosure or a Positive Control (PCtrl: sodium 2-(2-hydroxy-3,5-dipentylphenyl)acetate) as per the following. Fertilized eggs from white leghorn chicken were used and incubated in an Ova-Easy egg incubator at 37° C. with 60% humidity. At day 3, eggs were cracked. At day 9, U87 (human glioblastoma) cell suspensions (1×10⁶ cells), or Caki cells or patient-derived xenograph (glioblastoma fragment) were mixed (1:1) with growth factor reduced Matrigel™ in a total volume of 20 μl for cell lines and POX-patient fragments were placed directly on top of the CAM and were returned to the incubator. Intravenous injection at day 11 or topical administration (Day 11 to Day 16) with or without the compounds. At day 16, chick embryos were killed by decapitation. Tumours were removed and tumour volumes were calculated using the formula: (Dd²/3).

Table 4 summarizes the anticancer activity of these representative compounds on human glioblastoma U87 cell, human renal carcinoma Caki cell lines, glioblastoma from patient derived xenograft (POX) and human fibrosarcoma HT1080 cell. As shown, alcohol derivatives strongly improve anticancer activity relatively to their respective positive control compound (Pctrl or Setogepram).

TABLE 4

| | | Anticancer Activity | | | |
|---|---|---|---|---|---|
| | | | PDX | | |
| Compound | Structure | U87 | (Glioblastoma) | Caki | HT1080 |
| PCtrl | 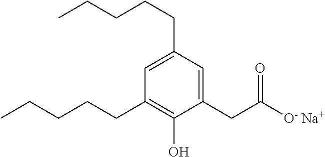 | X | | | |
| 1 | 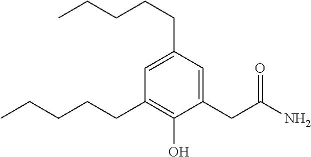 [SMILES: CCCCCc1cc(CCCCC)c(O)c(CC(N)=O)c1] | XX | | | |
| 2 | 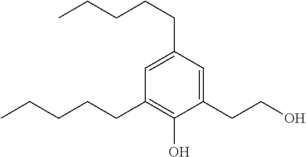 [SMILES: CCCCCc1cc(CCO)c(O)c(CCCCC)c1] | XXX | XX | XXXX | |
| 3 | 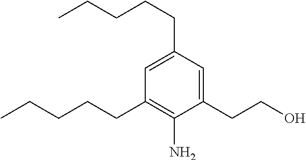 [SMILES: CCCCCc1cc(CCO)c(N)c(CCCCC)c1] | XX | | | |
| 4 | 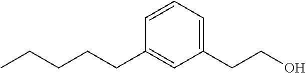 | | | | XXX |
| 5 | 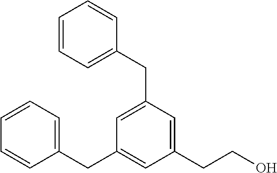 [SMILES: OCCc1cc(Cc2ccccc2)cc(Cc2ccccc2)c1] | XXXX | | XXXX | XXX |
| Setogepram | | | | | XX |

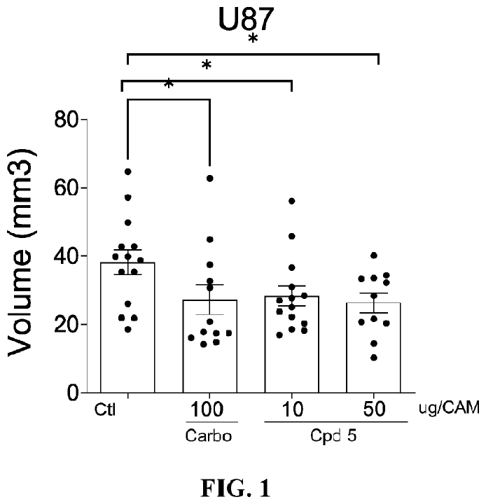

FIG. 1 illustrates the activity of Representative Compound 5 that reached the anticancer activity of carboplatin, a well-known alkylating agent used in chemotherapy, by a different mechanism targeting epithelial-mesenchymal transition (EMT), metabolism and fibrosis-related to cancer.

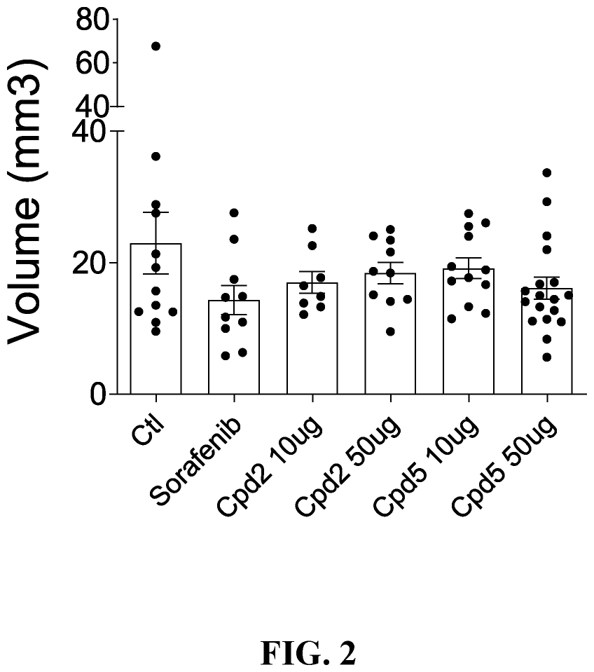

FIG. 2 illustrates the anticancer activity of Representative Compounds 2 and 5 on renal carcinoma Caki cells in CAM Avatar model. Representative Compounds 2 and 5 reach the anticancer activity of Sorafenib, a kinase inhibitor drug approved for the treatment of primary kidney cancer.

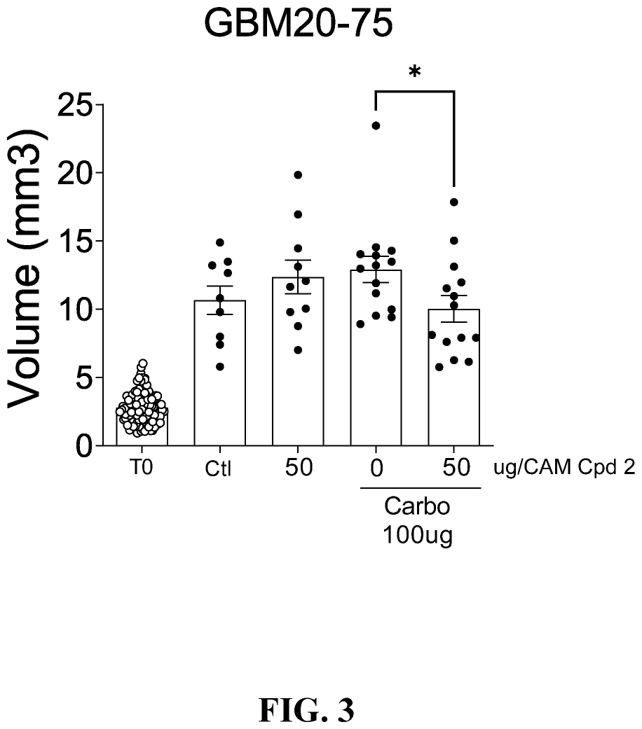

FIG. 3 illustrates the synergistic anticancer activity observed with the use of chemotherapy and Representative Compound 2 in a patient derived xenograft (PDX) glioblastoma resistant to carboplatin. As observed, the PDX cancer fragment increases in volume from the day of implantation (T0) to the day of treatment (Ctl). The results confirm that the PDX is effectively resistant to carboplatin and also does not respond to any treatments (Temozolomide (data not shown)) and Representative Compound 2. However, when treated with a combination of carboplatin and Representative Compound 2, a synergistic activity is observed and the combination treatment reaches a significant reduction of growth of a carboplatin-resistant PDX-glioblastoma.

### Example 3: Antiproliferative Effect of Compounds on Cancer Cells

Antiproliferative activity of representative compounds on cancer cells was also undertaken by using PC-3 cells (human prostate cancer cells). PC-3 cells were cultured for 24 hours in 96-well plates with or without Compounds or Positive Control (PCtrl: sodium 2-(2-hydroxy-3,5-dipentylphenyl)acetate) or Setogepram CAS No.: 1002101-19-0 from MedChemExpress LLC, USA) at various concentrations. The last four hours of incubation, 50 μl of a freshly made solution of MTT at 2 mg/ml in PBS were added. Cells were harvested, 150 μl of DMSO were added to solubilize the formazan crystal formed and the plates were read the plate at 570 nm.

Table 5 summarizes the percentage of inhibition of PC-3 cells proliferation by representative compounds. As shown, alcohol derivatives strongly improve antiproliferative/anticancer activity relatively to the positive control compound.

TABLE 5

| | | | | | | | | | Cpd4 | Setogepram |
|---|---|---|---|---|---|---|---|---|---|---|
| | | PCtrl | Cpd1 | Cpd2 | Cpd3 | Cpd5 | | | % of inhibition of | |
| Concentration | | % of inhibition of Proliferation | | | | | Concentration | | Proliferation | |
| 200 | uM | 20.0 | 0.0 | 66.3 | 65.8 | 53.0 | 1000 | uM | 70.4 | 0.0 |
| 100 | uM | 0.0 | 0.0 | 72.2 | 62.1 | 72.2 | 500 | uM | 72.9 | 0.0 |
| 50 | uM | 0.0 | 0.0 | 70.0 | 67.4 | 70.6 | 250 | uM | 74.2 | 10.4 |
| 25 | uM | 0.0 | 0.0 | 63.6 | 39.1 | 42.8 | 125 | uM | 30.3 | 5.7 |
| 12.5 | uM | 0.0 | 0.0 | 0.0 | 0.0 | 1.7 | 62.5 | uM | 26.1 | 16.8 |

### Example 4: Antifibrotic Effect of Compounds on Patient-Derived Xenograft IPF Lungs

Avatar CAM model was also used to determine the antifibrotic activity of the representative compounds. Briefly, fertilized eggs from white leghorn chicken were used and incubated in an Ova-Easy egg incubator at 37° C. with 60% humidity. At day 3, eggs were cracked. At day 9, PDX-fragments from IPF lungs were grafted on top of the CAM and were returned to the incubator. Treatment by intravenous injection of compounds was performed at day 11 and at day 16, chick embryos were killed by decapitation. IPF-PDX fragments were removed and volumes were calculated using the formula: (Dd²/3). Fibrosis was determined by a Masson's trichrome staining.

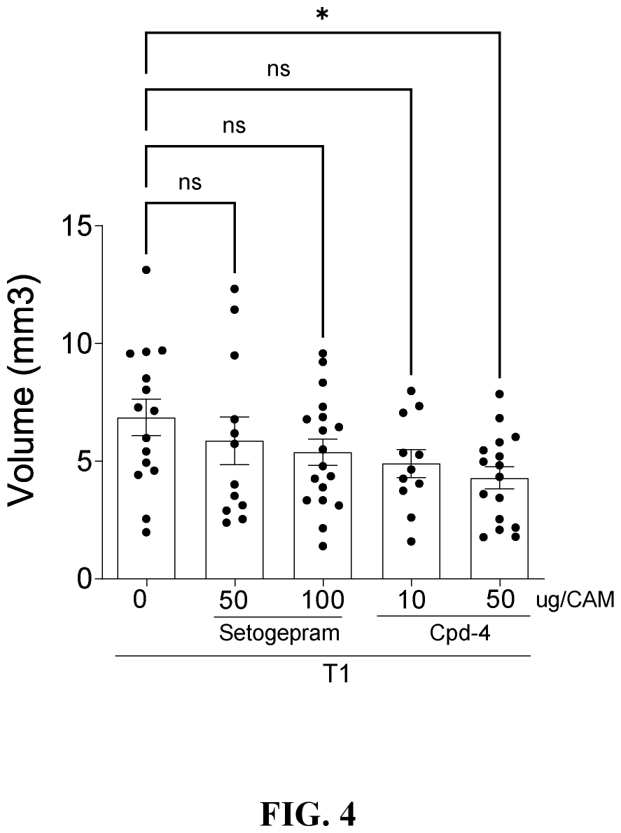

Table 6 shows antifibrotic activity of preferred compounds on PDX-IPF as shown by a reduction of the volume growth of the PDX-IPF-fragments. Results are compared to setogepram, a well-known antifibrotic compound and it shows that the alcohol derivative (Representative Compound 4) shows unexpected greater antifibrotic activity. As illustrated in FIG. 4, only Representative Compound 4 reduces significantly the volume of the PDX-IPF fragments, but both compounds reduce collagen deposition in the PDX-IPF fragment.

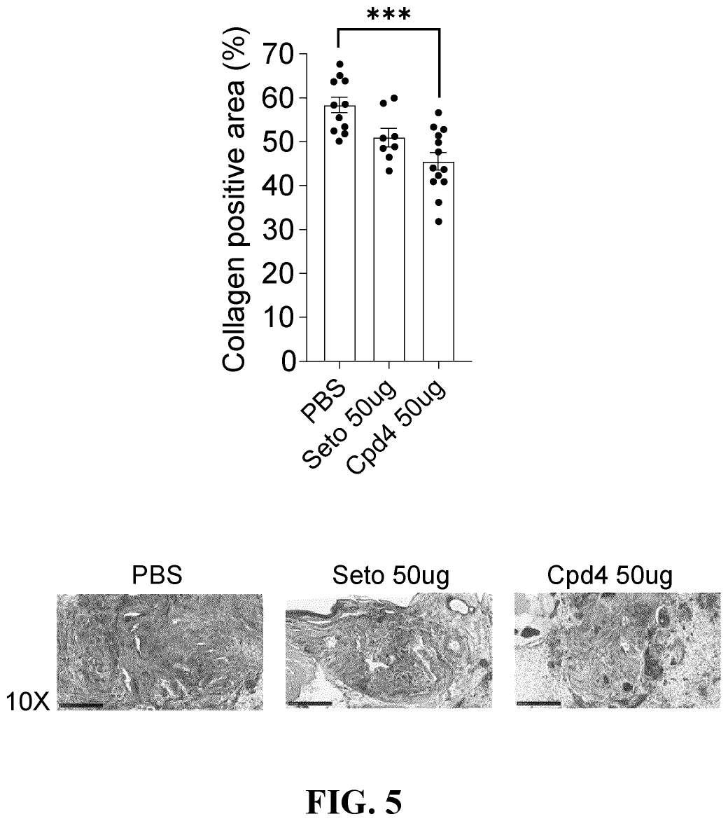

As illustrated in FIG. 5, both compounds, Compound 6 and setogepram, reduced the collagen deposition (blue) as determined by a Masson's trichrome staining, however the alcohol derivative achieved a very significant reduction of collagen in the PDX-IPF fragments.

TABLE 6

| | Antifibrotic |
|---|---|
| | Activity |
| Compound | PDX (IPF lungs) |
| Representative | XXXX |
| compound 4 | |
| Representative | XX |
| compound 5 | |
| Setogepram | XX |

### Example 5: Anti-Inflammatory/Antifibrotic/Metabolic/Analgesic Effect of Representative Compounds Determined by Cytokine Release from Human Peripheral Blood Mononuclear Cells (PBMC)

The anti-inflammatory/antifibrotic/metabolic and analgesic activity of Representative Compounds was undertaken with the analysis of cytokine releases under inflammatory conditions in LPS-stimulated PBMC.

It is well known that IL-6, MCP1, TNFα and IL-1β are pleiotropic cytokines involved in inflammation, fibrosis, metabolism and pain. Human peripheral blood mononuclear cells were isolated from venous blood of healthy volunteers by dextran sedimentation followed by centrifugation over Ficoll-Hypaque, according to the manufacturer's protocol. Freshly isolated human PBMC (4×10⁶ cells/mL suspended in RPMI-1640) were stimulated with or without LPS or Representative Compounds or Positive Control (PCtrl: sodium 2-(2-hydroxy-3,5-dipentylphenyl)acetate) or Setogepram (a well-know anti-inflammatory/antifibrotic agent) at various concentration and incubated for 4 and 24 h. After incubation, supernatants were collected and IL-6, MCP1, TNFα and IL-1β were measured by ELISA, as recommended by the manufacturer's protocol.

### IL-6

II-6 is a pleiotropic cytokine and functions as a proinflammatory factor as well as a profibrotic factor. Inflammatory/fibrotic models of chronic diseases and clinical observations identify also IL-6 activity as detrimental in autoimmunity and cancer. IL-6 plays also an important role in various metabolic processes as an autocrine and/or paracrine actions of adipocyte function. At present, accumulating evidence has demonstrated that IL-6 is closely linked to metabolic disorders such as MS and type 2 diabetes. IL-6 is also involved in the process of pathological pain.

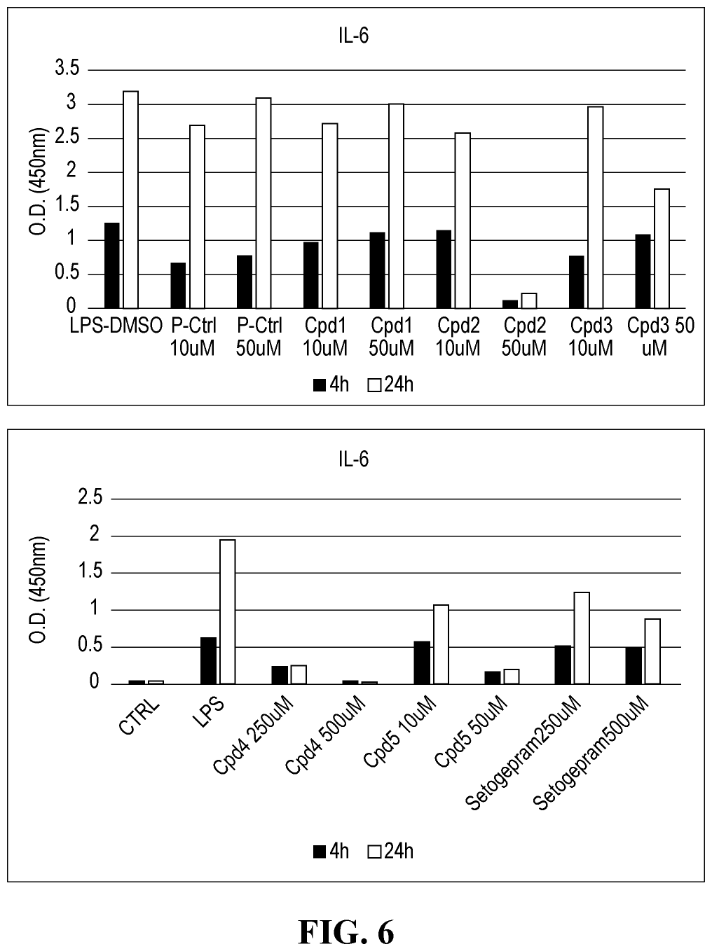

FIG. 6 represents the anti-inflammatory/antifibrotic/metabolic/analgesic activities of Representative Compounds as observed by a reduction of IL-6 release from LPS-stimulated PBMC. As observed previously, unexpected stronger inhibition is observed in alcohol derivatives.

### MCP-1

CCL2 is produced by many cell types, including endothelial, fibroblasts, epithelial, smooth muscle, mesangial, astrocytic, monocytic, and microglial cells. These cells are important for antiviral immune responses in the peripheral circulation and in tissues. However, monocyte/macrophages are found to be the major source of CCL2. CCL2 regulates the migration and infiltration of monocytes, memory T lymphocytes, and natural killer (NK) cells. CCL2 has been shown to be a potential intervention point for the treatment of various inflammatory and fibrotic diseases (IPF), as well as multiple sclerosis (Sorensen et al., Chemokine CCL2 and chemokine receptor CCR2 in early active multiple sclerosis. Eur J Neurol. 2004; 11:445-449), rheumatoid arthritis, atherosclerosis, and insulin-resistant diabetes.

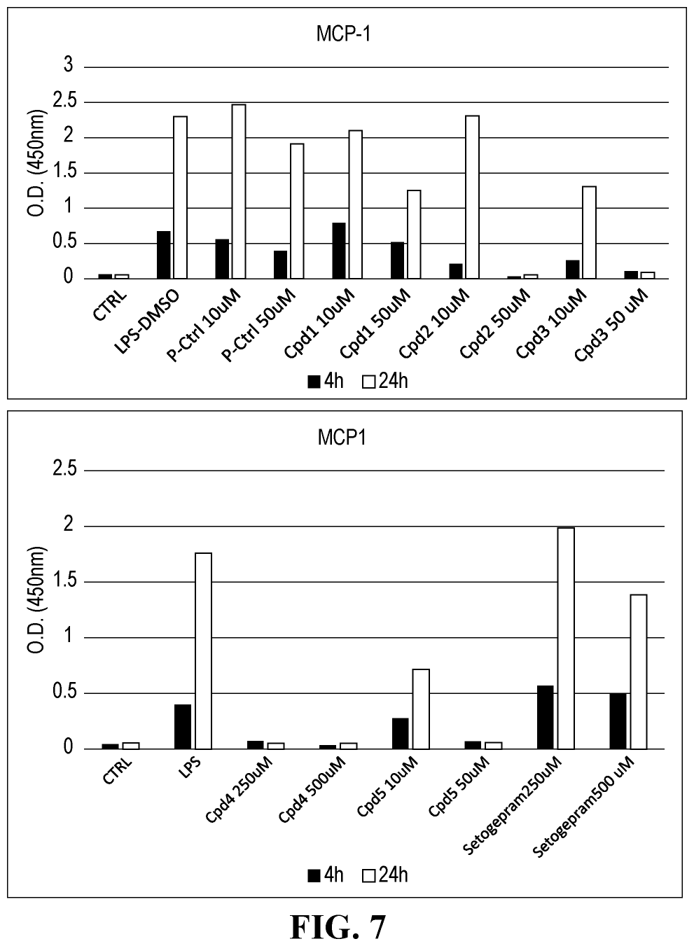

FIG. 7 represents the anti-inflammatory/antifibrotic/metabolic/analgesic activities of Representative Compounds as observed by a reduction of MCP-1 release from LPS-stimulated PBMC. As observed previously, unexpected stronger inhibition is observed in alcohol derivatives.

### TNFα

TNFα plays an important role in proinflammatory response and cell-to-cell communication. TNFα signaling is closely associated with various autoimmune and inflammatory diseases. Until now, five drugs targeting TNF have been developed: infliximab, etanercept, adalimumab, glomumab, and certolizumab pegol. The indications of these TNF-targeting drugs have been approved for rheumatoid arthritis, psoriatic arthritis, psoriasis, ankylosing spondylitis, juvenile idiopathic arthritis, Crohn's disease, and ulcerative colitis. TNF-α is also involved in the evolution of lung and liver fibrosis. Furthermore, TNFα seems to be increased in obese subjects, suggesting its role as a proinflammatory cytokine to insulin resistance and metabolic abnormalities in obesity.

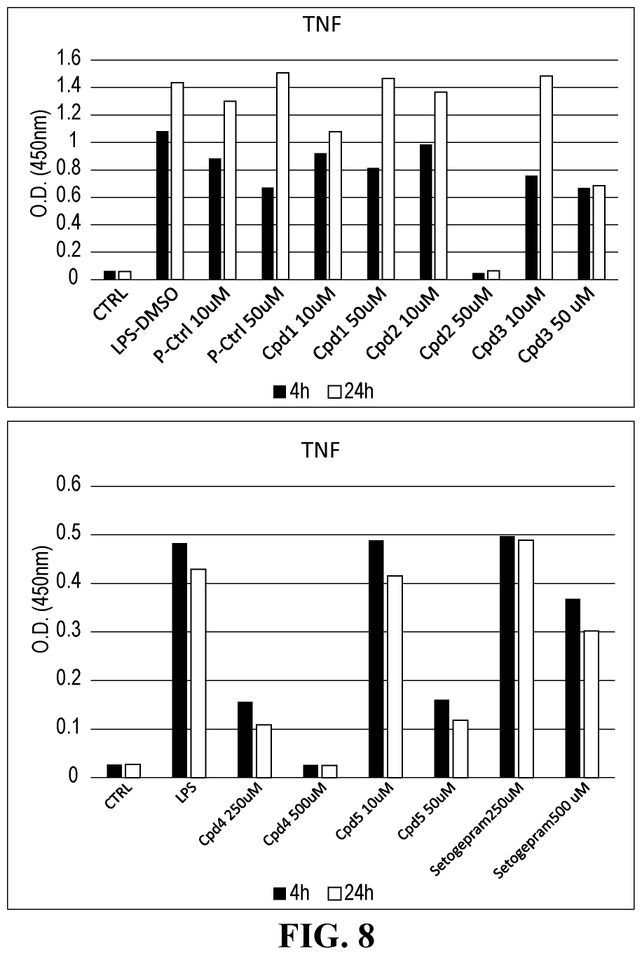

FIG. 8 represents the anti-inflammatory/antifibrotic/metabolic/analgesic activities of Representative Compounds as observed by a reduction of TNFα release from LPS-stimulated PBMC. As observed previously, unexpected stronger inhibition is observed in alcohol derivatives.

### IL-1β

IL-1β is an inducible cytokine and is not generally expressed in healthy cells or tissue; however, full-length IL-1β is rapidly induced in cells by activation of pattern recognition receptors (PRRs) such as TLRs by pathogen products or factors released by damaged cells, leading to intracellular accumulation of the protein. IL-1β is released by lung macrophages, and stimulates fibroblasts to synthesize collagen and produce fibrin. IL-1β is also involved in inflammatory, fibrotic, metabolic syndrome and pathological pain and related diseases.

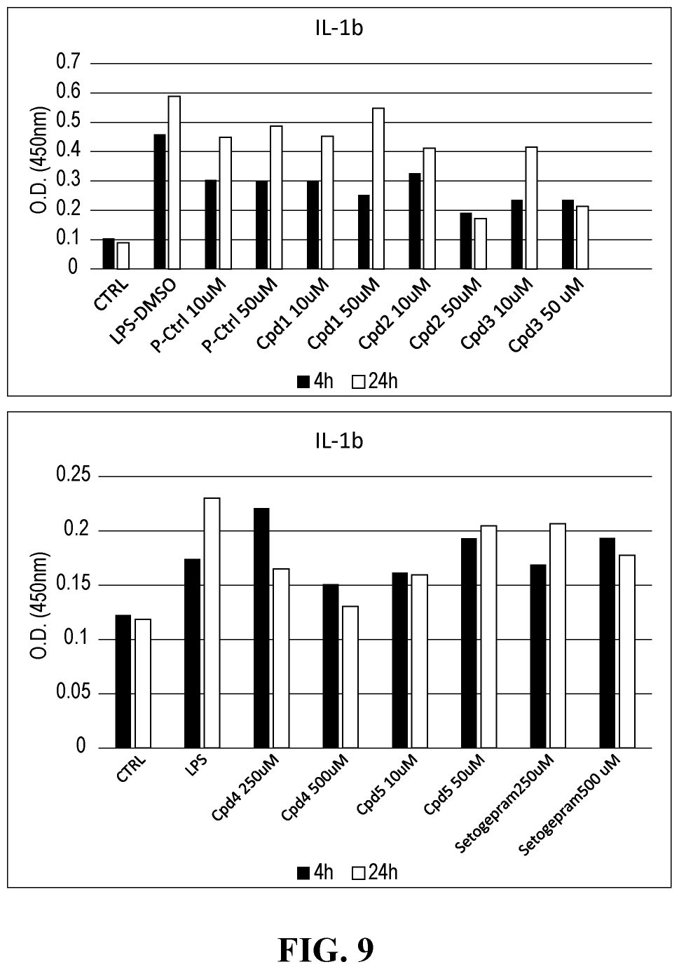

FIG. 9 represents the anti-inflammatory/antifibrotic/metabolic/analgesic activities of Representative Compounds as observed by a reduction of IL-1β release from LPS-stimulated PBMC. As observed previously, unexpected stronger inhibition is observed in alcohol derivatives.

Other examples of implementations will become apparent to the reader in view of the teachings of the present description and as such, will not be further described here.

The present results have shown that alcohol, aldehyde, amine, amide, ester, ether, lactone and/or ketone forms of substituted aromatic compounds (i.e., Formula I) as well as acceptable salt thereof provide compounds having advantageous properties. In particular, alcohol forms have demonstrated superior biological activity in the tests described herein compared to carboxylic acid forms (or acceptable salt thereof) of such substituted aromatic compounds.

Note that titles or subtitles may be used throughout the present disclosure for convenience of a reader, but in no way these should limit the scope of the present disclosure. Moreover, certain theories may be proposed and disclosed herein; however, in no way they, whether they are right or wrong, should limit the scope of the present disclosure so long as the invention is practiced according to the present disclosure without regard for any particular theory or scheme of action.

All references cited throughout the specification are hereby incorporated by reference in their entirety for all purposes.

Reference throughout the specification to “some embodiments”, and so forth, means that a particular element (e.g., feature, structure, and/or characteristic) described in connection with the invention is included in at least one embodiment described herein, and may or may not be present in other embodiments. In addition, it is to be understood that the described inventive features may be combined in any suitable manner in the various embodiments.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art in light of the present description that numerous modifications and variations can be made. The scope of the present invention is defined more particularly in the appended claims.

1. A compound according to Formula I having a core aromatic group with substituents as follows: 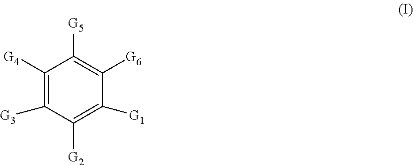
wherein
G₁ is —(CH₂)ₙC(R₁)(R₂)OH, —(CH₂)ₙ—CHO, —(CH₂)ₙC(O)NR₁R₂, —(CH₂)ₙCH (R₁)NR₁R₂, —(CH₂)ₙC(O)OR₃, —(CH₂)ₙ—CH(R₁)O—R₃, or —(CH₂)ₙC(O)R₃;
G₂ is H, NH₂, OH, F, or Cl;
G₃ is H, F, Cl, OH, —(CH₂)ₙ-optionally substituted heterocycle, —(CH₂)ₙ-optionally substituted phenyl, —(CH₂)ₙC₃H₅, optionally substituted C₁-C₆ alkyl, optionally substituted C₂-C₆ alkenyl, —C(O)—R₃, and CH(OH)—R₃;
G₄ is H, OH, F or Cl;
G₅ is H, OH, F, Cl, —(CH₂)ₙ-optionally substituted heterocycle, —(CH₂)ₙ-optionally substituted phenyl, —(CH₂)ₙC₃H₅, optionally substituted C₁-C₆ alkyl, optionally substituted C₂-C₆ alkenyl, —C(O)—R₃, or CH(OH)—R₃; and
G₆ is H, F, Cl, OH, —(CH₂)ₙ-optionally substituted heterocycle, or —(CH₂)ₙ-optionally substituted phenyl,
wherein
n is an integer selected from 0 to 5;
R₁ and R₂ are independently selected from H and optionally substituted C₁-C₆ alkyl group, and
R₃ is an optionally substituted C₁-C₆ alkyl group or when present on G₁ forms a lactone with the core aromatic group,
or a pharmaceutically acceptable salt thereof.

2. The compound or pharmaceutically acceptable salt thereof of claim 1, wherein G₃ is C₅ alkyl, C₅ alkenyl, —C(O)—(CH₂)₃—CH₃ or —CH(OH)—(CH₂)₃—CH₃.

3. The compound or pharmaceutically acceptable salt thereof of claim 1 wherein G₃ is C₆ alkyl, C₆ alkenyl, —C(O)—(CH₂)₄—CH₃ or —CH(OH)—(CH₂)₄—CH₃.

4. The compound or pharmaceutically acceptable salt thereof of claim 1, wherein G₃ is C₅ alkyl, C₆ alkyl, C₅ alkenyl, or C₆ alkenyl.

5. The compound or pharmaceutically acceptable salt thereof of claim 1, wherein G₃ is C₅ alkyl or C₅ alkenyl.

6. The compound or pharmaceutically acceptable salt thereof of claim 1, wherein G₃ is C₅ alkyl or C₆ alkyl.

7. The compound or pharmaceutically acceptable salt thereof of claim 1, wherein G₃ is C₅ alkyl.

8. The compound or pharmaceutically acceptable salt thereof of claim 1, wherein G₃ is —(CH₂)ₙ-optionally substituted phenyl.

9. The compound or pharmaceutically acceptable salt thereof of claim 8, wherein the phenyl is substituted with an optionally substituted C₁-C₆ alkyl.

10. The compound or pharmaceutically acceptable salt thereof of claim 9, wherein G₃ is CH₃(CH₂)ₓ—C₆H₄—(CH₂)_{y}— wherein x+y=4 or 5, and wherein y is an integer selected from 0 to 5.

11. The compound or pharmaceutically acceptable salt thereof of claim 1, wherein G₃ is —(CH₂)ₙ-optionally substituted heterocycle.

12. The compound or pharmaceutically acceptable salt thereof of claim 11, wherein the heterocycle has from 1 to 3 heteroatoms selected from nitrogen, oxygen, and sulfur.

13. The compound or pharmaceutically acceptable salt thereof of claim 11, wherein the heterocycle is a non-aromatic monocyclic or polycyclic ring.

14. The compound or pharmaceutically acceptable salt thereof of claim 11, wherein the heterocycle is an aromatic ring.

15. The compound or pharmaceutically acceptable salt thereof of claim 1, wherein G₅ is H, OH, F, —CH₂Phe, —CH₂—C₃H₅, C₄-C₆ alkyl, —(CH₂)ₙCH═CH, or —CH═CH(CH₂), wherein n is 2 or 3.

16. The compound or pharmaceutically acceptable salt thereof of claim 1, wherein G₁ is
—(CH₂)ₙCH(CH₃)OH;
—(CH₂)ₙ—CH₂—O—CH₃;
—(CH₂)ₙCH(O)NH₂;
—(CH₂)ₙC(O)R₃;
—C(CH₃)₂OH;
—CH(F)—OH;
—CF₂—OH;
—C(O)CH₃;
—CH₂C(O)OR₃; or
—(CH₂)ₙC(O)R₃;
or pharmaceutically acceptable salt thereof.

17. The compound or pharmaceutically acceptable salt thereof of claim 1, wherein G₁ is —(CH₂)ₙC(R₁)(R₂)OH.

18. The compound or pharmaceutically acceptable salt thereof of claim 1, wherein G₁ is —(CH₂)ₙ—CHO.

19. The compound or pharmaceutically acceptable salt thereof of claim 1, wherein G₁ is —(CH₂)ₙC(O)NR₁R₂.

20. The compound or pharmaceutically acceptable salt thereof of claim 1, wherein G₁ is —(CH₂)ₙCH (R₁)NR₁R₂.

21. The compound or pharmaceutically acceptable salt thereof of claim 1, wherein G₁ is —(CH₂)ₙC(O)OR₃.

22. The compound or pharmaceutically acceptable salt thereof of claim 1, wherein G₁ is —(CH₂)ₙ—CH(R₁)O—R₃.

23. The compound or pharmaceutically acceptable salt thereof of claim 1, wherein G₁ is —(CH₂)ₙC(O)R₃.

24. The compound or pharmaceutically acceptable salt thereof of claim 1, wherein the compound is:
2-(2-hydroxypropyl)-4,6-dipentylphenol;
4-benzyl-2-(2-hydroxypropyl)-6-pentylphenol;
2,4-dibenzyl-6-(2-hydroxypropyl)phenol;
2-benzyl-6-(2-hydroxypropyl)-4-pentylphenol;
2,4-bis(3-cyclopropylpropyl)-6-(2-hydroxypropyl)phenol;
2-(2-hydroxy-3,5-dipentylphenyl)acetamide;
2-(5-benzyl-2-hydroxy-3-pentylphenyl)acetamide;
2-(3,5-dibenzyl-2-hydroxyphenyl)acetamide;
2-(3-benzyl-2-hydroxy-5-pentylphenyl)acetamide;
2-(3,5-bis(3-cyclopropylpropyl)-2-hydroxyphenyl)acetamide;
2-(2-hydroxy-3,5-dipentylphenyl)acetaldehyde;
2-(5-benzyl-2-hydroxy-3-pentylphenyl)acetaldehyde;
2-(3,5-dibenzyl-2-hydroxyphenyl)acetaldehyde;
2-(3-benzyl-2-hydroxy-5-pentylphenyl)acetaldehyde;
2-(3,5-bis(3-cyclopropylpropyl)-2-hydroxyphenyl)acetaldehyde;
1-(2-hydroxy-3,5-dipentylphenyl)propan-2-one;
1-(5-benzyl-2-hydroxy-3-pentylphenyl)propan-2-one;
1-(3,5-dibenzyl-2-hydroxyphenyl)propan-2-one;
1-(3-benzyl-2-hydroxy-5-pentylphenyl)propan-2-one;
1-(3,5-bis(3-cyclopropylpropyl)-2-hydroxyphenyl)propan-2-one;
methyl 2-(2-hydroxy-3,5-dipentylphenyl)acetate;
methyl 2-(5-benzyl-2-hydroxy-3-pentylphenyl)acetate;
methyl 2-(3,5-dibenzyl-2-hydroxyphenyl)acetate;
methyl 2-(3-benzyl-2-hydroxy-5-pentylphenyl)acetate;
methyl 2-(3,5-bis(3-cyclopropylpropyl)-2-hydroxyphenyl)acetate;
2-(2-methoxypropyl)-4,6-dipentylphenol;
4-benzyl-2-(2-methoxypropyl)-6-pentylphenol;
2,4-dibenzyl-6-(2-methoxypropyl)phenol;
2-benzyl-6-(2-methoxypropyl)-4-pentylphenol; or
2,4-bis(3-cyclopropylpropyl)-6-(2-methoxypropyl)phenol,
or pharmaceutically acceptable salt thereof.

25. The compound or pharmaceutically acceptable salt thereof of claim 1, which is: 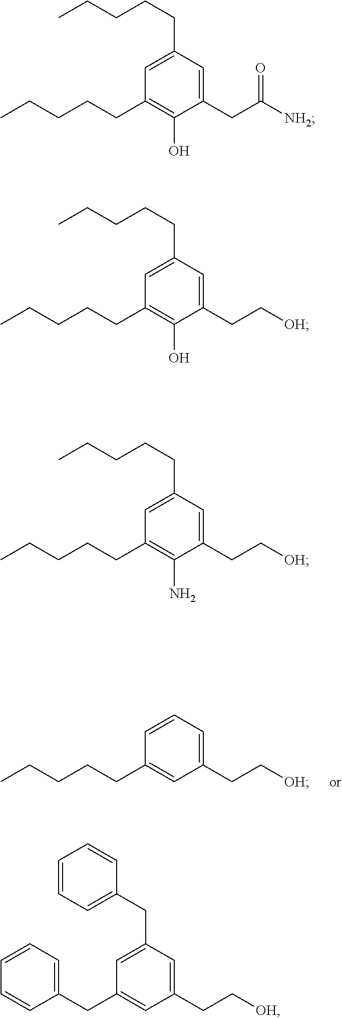 [SMILES: CCCCCc1cc(CCCCC)c(O)c(CC(N)=O)c1 ; CCCCCc1cc(CCO)c(O)c(CCCCC)c1 ; CCCCCc1cc(CCO)c(N)c(CCCCC)c1 ; CCCCCc1cccc(CCO)c1 ; OCCc1cc(Cc2ccccc2)cc(Cc2ccccc2)c1]
or pharmaceutically acceptable salt thereof.

26. The compound or pharmaceutically acceptable salt thereof of claim 1, wherein the pharmaceutically acceptable salt of said compound is an organic or inorganic salt.

27. The compound or pharmaceutically acceptable salt thereof of claim 26, wherein the salt is a sodium, potassium, lithium, ammonium, calcium, magnesium, manganese, zinc, iron, or copper salt.

28. The compound or pharmaceutically acceptable salt thereof of claim 26, wherein the salt is an acetate, benzoate, besylate, bromide, carbonate, citrate, edisylate, estolate, fumarate, gluconate, hippurate, iodide, maleate, mesylate, methylsulfate, napsylate, oxalate, pamoate, phosphate, stearate, succinate, sulfate, tartrate, tosylate, or chloride salt.

29.-43. (canceled)

44. A pharmaceutical composition comprising the compound or pharmaceutically acceptable salt thereof of claim 1 and a pharmaceutical acceptable carrier, diluent or carrier.

45. A method for treatment or prevention of cancer, inflammatory-related disease, oxidative stress, pain, metabolic disorder or a fibrotic-related disease in a subject, comprising administering a therapeutically effective amount of the compound of claim 1 to the subject.

46. (canceled)

47. (canceled)

48. The method of claim 45, wherein the treatment of cancer includes inhibition of tumor growth, cell proliferation, tumor cell migration or metastasis in a subject suffering from the cancer.

49. (canceled)

50. (canceled)

51. (canceled)

52. The method of claim 45, wherein the compound of claim 1 is administered in combination with an anticancer agent.

53. The method of claim 52, wherein the anticancer agent is temozolomide, abraxane, decarbazine, doxorubicin, daunorubicin, cyclophosphamide, busulfex, busulfan, bleomycin, alectinib, melphalan, pamidronate, bevacizumab, carbozantinib, vinblastine, docetaxel, prednisolone, ifosphamide, dexamethasone, vincristine, bleomycin, etoposide, topotecan, mitomycine, irinotecan, taxotere, taxol, 5-fluorouracil, folfirinox, methotrexate, gemcitabine, cisplatin, carboplatin, chlorambucil, beribucin, or tyrosine kinase inhibitors.

54. The method of claim 45, wherein the cancer is bladder cancer, breast cancer, colorectal cancer, kidney cancer, melanoma, non-Hodgkin's lymphoma, lung, liver, leukemia, glioblastoma, ovarian cancer, pancreatic cancer, prostate cancer or uterine cancer.

55. The method of claim 45, wherein the cancer is glioblastoma or melanoma, and wherein the compound is administered in combination with chitosan for in situ treatment of recurrence of cancer.

56. The method of claim 45, for treating or preventing a fibrotic-related disease.

57. (canceled)

58. (canceled)

59. (canceled)

60. The method of claim 45, wherein the subject is a human.
